(19) United States
(12) Patent Application Publication
(10) Pub. No.: US 2022/0057398 A1
(43) Pub. Date: Feb. 24, 2022

(54) BIOMARKERS FOR TYPE 1 DIABETES

(71) Applicant: Board of Regents of the University of Nebraska, Lincoln, NE (US)

(72) Inventors: Nora E. Sarvetnick, Omaha, NE (US); Robert Z. Harms, Omaha, NE (US)

(73) Assignee: Board of Regents of the University of Nebraska, Lincoln, NE (US)

(21) Appl. No.: 17/250,844

(22) PCT Filed: Sep. 11, 2019
(86) PCT No.: PCT/US19/50671

§ 371 Date: Mar. 11, 2021

Related U.S. Application Data
(60) Provisional application No. 62/732,115, filed on Sep. 17, 2018.
(60) Provisional application No. 62/730,834, filed on Sep. 13, 2018.

(51) Int. Cl. G01N 33/569

(52) U.S. Cl. CPC G01N 33/56972; G01N 2800/042; G01N 2333/705

(57) ABSTRACT

In various embodiments methods are provided for evaluating the likelihood of a subject progressing to type 1 diabetes. In certain embodiments the methods involve determining the level of CD57+, CD28−, CD127−, CD27−, CD8+ T cells (SLEC CD8 T cells) and the other populations shown in FIG. 10 and/or CCR7^{dim}, CD45RA+, CD8+ T cells derived from a subject where an elevated level of the first populations above, and/or a reduced level of the CCR7^{dim}, CD45RA+, CD8+ T cells as compared to the level(s) in a normal healthy control is an indicator that subject has a significant elevated risk for progression to type I diabetes.

### CROSS-REFERENCE TO RELATED APPLICATIONS

This application claims priority to and benefit of U.S. Ser. No. 62/730,834, filed on Sep. 13, 2018 and to U.S. Ser. No. 62/732,115, filed on Sep. 17, 2018, both of which are incorporated herein by reference in their entirety for all purposes.

### STATEMENT OF GOVERNMENTAL SUPPORT

This invention was made with government support under Grants No. U01AI130841 awarded by the National Institutes of Health. The government has certain rights in the invention.

### BACKGROUND

Human type 1 diabetes (T1D) is characterized by the presence of antibodies targeting islet cell-associated antigens (i.e. autoantibodies) and the selective destruction of beta cells resulting in insulin deficiency (Atkinson et al. (2017) The Lancet 383: 69-82). The destruction of beta cells is widely believed to be driven by autoreactive T cells (Puglies (2017) J. Clin. Invest. 127: 2881-2891). The evidence for this theory is generally indirect, comprising multiple reports of a variable T cell presence in and around islets (see, e.g., Willcox et al. (2009) Clin. & Exp. Immunol. 155: 173-181) including islet-antigen reactive CD8 T cells (Coppieters et al. (2012) J. Exp. Med. 209(1): 51-60). Furthermore, islet-antigen reactive CD8 T cells have been isolated from the peripheral blood of type 1 diabetics and these can be induced to destroy beta cells in culture (Skowera et al. (2008) J. Clin. Invest. 18: 3390-3402). In vivo. this unimpeded destruction by autoreactive CD8 T cells is considered to indicate an escape from tolerance. possibly due to dysfunctional regulatory T cells (Treg) among diabetics (Hull et al. (2017) Diabetologia 60: 1839-1850).

Defining the prodromal events that ultimately manifest in clinically-defined T1D remains a critical area of research. It is known that autoantibody presence often occurs several years prior to diagnosis (Steck et al. (2011) Diabetes Care, 34: 1397-1399) with functional aberrancy and reduction in the beta cell population preceding overt hyperglyceamia (Chen et al. (2017) Mol. Metab. 6: 943-2957; Wilcox et al. (2016) J. Autoimmun. 71: 51-58). While genetic factors can identify some risk. environmental variables are the ostensible drivers of T1D and its rising rate of incidence (Mayer-Davis et al. (2017) New Engl. J. Med. 376: 1419-14291 Pociot & Lernmark (2016) The Lancet, 387: 2331-2339; Rewers & Ludvigsson (2016) The Lancet, 387: 2340-2348), in the absence of a well-characterized environmental trigger that may be avoided, early intervention following seroconversion is a worthy clinical goal for at-risk subjects (Rewers & Gottlieb (2009) Diabetes Care, 32: 1769-1782). To that end, a thorough investigation of immune cells during the preclinical period is warranted. Such an investigation can reveal immune perturbations indicative of environmental exposures and/or regulatory dysfunction. These data can then be used to define biomarkers indicating risk. in the design of therapeutics, and in the unraveling of autoimmune mechanisms leading to beta cell decline. Although a simply stated need, the identification of at-risk subjects with subsequent leukocyte isolation is a near Herculean labor.

### SUMMARY

An ongoing collaboration was established to meet this challenge. The T1D TrialNet Pathway to Prevention Study involves screening relatives of type 1 diabetics for autoantibodies to gauge risk, as well as a component for biosample acquisition (Battaglia et al. (2017) Diabetologia, 60(11): 2139-2147). This collaboration has already yielded several important observations. We utilized TrialNet PBMC samples to interrogate multiple T cell populations using flow cytometry. Our results demonstrate profound changes in frequency of T cell subsets are associated with seroconversion. with several T cell lineages impacted, including helper (CD4). Regulatory-like (Treg-like), cytotoxic (CD8), and mucosal associated invariant T (MAIT) cells. These data suggest substantial immunodeficiency underrides a likely pathogen response among seroconverted subjects.

More specifically, it was discovered that elevated levels of CD57+, CD28−, CD127−, CD27−, CD8+ T cells (SLEC CD8 T cells) and/or elevated levels of CCR7^{dim}, CD45RA+, CD8+ T cells are indicative of likely progression to type 1 diabetes (T1D).

Accordingly, various embodiments contemplated herein may include, but need not be limited to, one or more of the following:

Various embodiments cnetemplated herein may include, but need not be limited to, one or more of the following:

Embodiment 1: A method of determining if a subject is at risk for progression to type 1 diabetes, said method comprising:
determining the level(s) of CD57+, CD28−, CD127−, CD27−, CD8+T cells (SLEC CD8 T cells), and/or CD127−, CD27+, CD57−, CD28− CD8 T cells, and/or CD127−, CD27−, CD57−, CD28− CD8 T cells, and/or CD127+, CD27−, CD57−, CD28− CD8 T cells, and/or associated CD8 T cell subsets shown in FIG. 10, and/or CCR7^{dim}, CD45RA+, CD8+ T cells in blood or a blood fraction derived from said subject, where:
an elevated level of CD57+, CD28−, CD127−, CD27−, CD8+ T cells (SLEC CD8 T cells), and/or CD127−, CD27+, CD57−, CD28− CD8 T cells, and/or CD127−, CD27−, CD57−, CD28− CD8 T cells, and/or CD127+, CD27−, CD57−, CD28− CD8 T cells, and/or associated CD8 T cell subsets shown in FIG. 10 as compared to the level(s) of said T cells in a normal healthy control that does not progress to type 1 diabetes is an indicator that said subject has a significant risk for progression to type 1 diabetes; and/or
a reduced level CCR7^{dim}, CD45RA+, CD8+ T cells as compared to the level of said CCR7^{dim}, CD45RA+, CD8+ T cells T cells in a normal healthy control that does not progress to type 1 diabetes is an indicator that said subject has a significant risk for progression to type 1 diabetes.

Embodiment 2: The method of embodiment 1, wherein said method comprises determining the level of CD57+, CD28−, CD127−, CD27−, CD8+ T cells (SLEC CD8 T cells) in blood or a blood fraction derived from said subject, where an elevated level of SLEC CD8 T cells as compared to the level of SLEC CD8 T cells in a normal healthy control that does not progress to type 1 diabetes is an indicator that said subject has a significant risk for progression to type 1 diabetes.

Embodiment 3: The method according to any one of embodiments 1-2, wherein said method comprises determining the level of CD127−, CD27+, CD57−, CD28− CD8 T cells in blood or a blood fraction derived from said subject, where an elevated level of said CD127−, CD27+, CD57−, CD28− CD8 T cells T cells as compared to the level of said CD127−, CD27+, CD57−, CD28− CD8 T cells in a normal healthy control that does not progress to type 1 diabetes is an indicator that said subject has a significant risk for progression to type 1 diabetes.

Embodiment 4: The method according to any one of embodiments 1-3, wherein said method comprises determining the level of CD127−, CD27−, CD57−, CD28− CD8 T cells in blood or a blood fraction derived from said subject, where an elevated level of said CD127−, CD27−, CD57−, CD28− CD8 T cells T cells as compared to the level of said CD127−, CD27−, CD57−, CD28− CD8 T cells in a normal healthy control that does not progress to type 1 diabetes is an indicator that said subject has a significant risk for progression to type 1 diabetes.

Embodiment 5: The method according to any one of embodiments 1-4, wherein said method comprises determining the level of CD127+, CD27−, CD57−, CD28− CD8 T cells in blood or a blood fraction derived from said subject, where an elevated level of said CD127+, CD27−, CD57−, CD28− CD8 T cells T cells as compared to the level of said CD127+, CD27−, CD57−, CD28− CD8 T cells in a normal healthy control that does not progress to type 1 diabetes is an indicator that said subject has a significant risk for progression to type 1 diabetes.

Embodiment 6: The method of according to any one of embodiments 1-5, wherein said method comprises determining the level of CCR7^{dim}, CD45RA+, CD8+ T cells in blood or a blood fraction from said subject, where a reduced level of said CD8 T cells as compared to the level of said T cells in a normal healthy control that does not progress to type 1 diabetes is a further indicator that said subject has a significant risk for progression to type 1 diabetes.

Embodiment 7: The method according to any one of embodiments 1-6, wherein said elevated level and/or said reduced level is a statistically significant elevated level and/or a statistically significant reduced level compared to said normal healthy control.

Embodiment 8: The method of embodiment 7, wherein said elevated level and/or said reduced level is a statistically significant elevated level and/or a statistically significant reduced level compared to said normal healthy control(s) at a p value of p≤0.05, or p≤0.02, or p≤0.01.

Embodiment 9: The method according to any one of embodiments 1-8, wherein the level(s) of CD57+, CD28−, CD127−, CD27−, CD8+ T cells (SLEC CD8 T cells), and/or CD127−, CD27+, CD57−, CD28− CD8 T cells, and/or CD127−, CD27−, CD57−, CD28− CD8 T cells, and/or CD127+, CD27−, CD57−, CD28− CD8 T cells, and/or associated CD8 T cell subsets shown in FIG. 10, and/or CCR7^{dim}, CD45RA+, CD8+ T cells are determined by antibody labeling of T cells and flow cytometry.

Embodiment 10: The method according to any one of embodiments 1-9, wherein said subject is a subject that has been identified as seroconverted for autoantibodies.

Embodiment 11: The method of embodiment 10, wherein said subject is identified as seroconverted by screening blood or a blood fraction from said subject for antibodies directed against one of more biomarkers selected from the group consisting of GAD65, IA-2, and IAA.

Embodiment 12: The method of embodiment 11, wherein after a positive identification of antibodies against one or more of said biomarkers, blood or a blood fraction derived from said subject is screened for the biomarkers ZnT8 and/or ICA.

Embodiment 13: The method according to any one of embodiments 11-12, wherein positive thresholds for said biomarkers are: IAA>0.010, GAD65>0.032, ICA512>0.049, GAD65H>20; IA-2H>5, ZnT8>0.020 and/or ICA≥10.

Embodiment 14: The method according to any one of embodiments 1-13, wherein said subject has not progressed to clinical type 1 diabetes.

Embodiment 15: The method of embodiment 14, wherein said subject shows a normal hemoglobin A1c (HbA1c) level ranging from 4% to 5.6%.

Embodiment 16: The method of embodiment 14, wherein said subject shows an elevated hemoglobin A1c (HbA1c) level ranging from 5.7% to 6.4%.

Embodiment 17: The method of embodiment 14, wherein a random blood sugar test for said subject is below 200 mg/dL (11.1 mmol/L).

Embodiment 18: The method of embodiment 14, wherein a normal fasting blood sugar level for said subject is less than 100 mg/dL (5.6 mmol/L).

Embodiment 19: The method of embodiment 14, wherein an elevated fasting blood sugar level for said subject ranges from 100 to 125 mg/dL (5.6 to 6.9 mmol/L).

Embodiment 20: The method according to any one of embodiments 1-19, wherein said subject is evaluated for the presence of an active viral infection.

Embodiment 21: The method of embodiment 20, where said subject is evaluated for an active viral infection by screening for viral IgM positivity, and/or analyzing plasma or circulating leukocytes for presence and abundance of viral nucleic acids.

Embodiment 22: The method according to any one of embodiments 20-21, wherein said viral infection is a CMV or other Herpesvirus infection.

Embodiment 23: The method according to any one of embodiments 1-22, wherein the level of CD57+, CD28−, CD127−, CD27−, CD8+ T cells (SLEC CD8 T cells), and/or CD127−, CD27+, CD57−, CD28− CD8 T cells, and/or CD127−, CD27−, CD57−, CD28− CD8 T cells, and/or CD127+, CD27−, CD57−, CD28− CD8 T cells, and/or associated CD8 T cell subsets shown in FIG. 10, and/or CCR7^{dim}, CD45RA+, CD8+ T cells and/or a diagnosis/prognosis based, at least in part, on said levels is recorded in a medical record for said subject.

Embodiment 24: The method according to any one of embodiments 1-23, wherein the level of level of CCR7^{dim}, CD45RA+, CD8+ T cells and/or a diagnosis/prognosis based, at least in part, on said levels is recorded in a medical record for said subject.

Embodiment 25: The method according to any one of embodiments 23-24, wherein said medical record is maintained by a laboratory, physician's office, a hospital, a health maintenance organization, an insurance company, or a personal medical record website.

Embodiment 26: The method according to any one of embodiments 1-25, wherein a diagnosis, based at least in part on the level(s) of CD57+, CD28−, CD127−, CD27−, CD8+ T cells (SLEC CD8 T cells), and/or CD127−, CD27+, CD57−, CD28− CD8 T cells, and/or CD127−, CD27−, CD57−, CD28− CD8 T cells, and/or CD127+, CD27−, CD57−, CD28− CD8 T cells, and/or associated CD8 T cell subsets shown in FIG. 10, and/or CCR7^{dim}, CD45RA+, CD8+ T cells is recorded on or in a medic alert article selected from a card, worn article, or radiofrequency identification (RFID) tag.

Embodiment 27: The method according to any one of embodiments 23-26, wherein the level(s) of CD57+, CD28−, CD127−, CD27−, CD8+ T cells (SLEC CD8 T cells), and/or CD127−, CD27+, CD57−, CD28− CD8 T cells, and/or CD127−, CD27−, CD57−, CD28− CD8 T cells, and/or CD127+, CD27−, CD57−, CD28− CD8 T cells, and/or associated CD8 T cell subsets shown in FIG. 10, and/or CCR7^{dim}, CD45RA+, CD8+ T cells and/or a diagnosis based upon said levels is recorded on a non-transient computer readable medium.

Embodiment 28: The method according to any one of embodiments 1-27, wherein the level(s) of CD57+, CD28−, CD127−, CD27−, CD8+ T cells (SLEC CD8 T cells), and/or CD127−, CD27+, CD57−, CD28− CD8 T cells, and/or CD127−, CD27−, CD57−, CD28− CD8 T cells, and/or CD127+, CD27−, CD57−, CD28− CD8 T cells, and/or associated CD8 T cell subsets shown in FIG. 10, and/or CCR7^{dim}, CD45RA+, CD8+ T cells are determined as part of a differential diagnosis.

Embodiment 29: The method according to any one of embodiments 1-28, wherein said subject is a human and said blood or blood fraction is from said human.

Embodiment 30: The method according to any one of embodiments 1-29, wherein when said subject is identified as at significant risk for progression to type 1 diabetes the subject is provided treatment to slow or prevent the onset of type 1 diabetes.

Embodiment 31: The method of embodiment 30, wherein said treatment comprises administering an immune modulator to said subject.

Embodiment 32: The method of embodiment 31, wherein said immune modulator comprises an anti-viral agent, an anti-PD1 antibody, an anti-CTLA4 antibody, and/or an anti-CD3 antibody.

Embodiment 33: The method of embodiment 32, wherein said immune modulator comprises an anti-CD3 monoclonal antibody.

Embodiment 34: The method of embodiment 33, wherein said immune modulator comprises teplizumab.

Embodiment 35: The method of embodiment 32, wherein said immune modulator comprises an anti-viral agent.

Embodiment 36: The method of embodiment 35, wherein said immune modulator comprises an anti-viral agent selected from the group consisting of Pleconaril and Ribavirin.

Embodiment 37: The method according to any one of embodiments 30-36, wherein said method comprises re-induction of tolerance towards the putative self-antigen that causes T1D.

Embodiment 38: The method of embodiment 37, wherein said antigen comprises one or more antigen selected from the group consisting of insulin, glutamic acid decarboxylase (GAD), and the heat shock protein 60 (Hsp60)-derived peptide 277.

Embodiment 39: The method according to any one of embodiments 30-38, wherein said treatment comprises monitoring blood glucose levels in said subject.

Embodiment 40: The method of embodiment 39, wherein said blood glucose is tested before meals and snacks, and/or before bed, and/or before exercising or driving.

Embodiment 41: The method of embodiment 39, wherein said blood glucose is monitored on a daily, a weekly, a biweekly, or a monthly basis.

Embodiment 42: The method according to any one of embodiments 30-41, wherein said treatment comprises fitting said subject with a continuous glucose monitoring system.

Embodiment 43: The method according to any one of embodiments 30-42, wherein said treatment comprises monitoring hemoglobin A1c.

Embodiment 44: The method according to any one of embodiments 30-43, wherein said treatment comprise maintaining normal blood glucose levels.

Embodiment 45: The method of embodiment 44, wherein said maintaining normal blood glucose levels comprises administering insulin.

Embodiment 46: The method of embodiment 45, wherein said insulin comprises an insulin selected from the group consisting of short-acting (regular) insulin, rapid-acting insulin, intermediate-acting (NPH) insulin, and long-acting insulin.

Embodiment 47: The method according to any one of embodiments 30-46, wherein said treatment comprises maintaining the subject on a low carbohydrate diet.

Embodiment 48: The method according to any one of embodiments 30-47, wherein said treatment comprise maintaining said subject on a diet that provides a BMI of said subject ranging from about 18.5 to about 24.9.

Embodiment 49: The method according to any one of embodiments 30-48, wherein said treatment comprise engaging said subject in daily exercise comprising at least 150 minutes of aerobic exercise a week, with no more than two days without any exercise.

Embodiment 50: The method according to any one of embodiments 30-49, wherein said treatment comprises providing said subject enzyme (ACE) inhibitors or angiotensin II receptor blockers (ARBs).

Embodiment 51: The method according to any one of embodiments 30-50, wherein said treatment comprises providing said subject aspirin.

Embodiment 52: The method according to any one of embodiments 30-51, wherein said treatment comprises providing said subject cholesterol lowering drugs.

Embodiment 53: A method of monitoring the onset and/or progression of type 1 diabetes in a subject, said method comprising:
providing a blood sample from said subject at a first time;
performing the method according to any one of embodiments 1-13 to determine first level(s) of CD57+, CD28−, CD127−, CD27−, CD8+ T cells (SLEC CD8 T cells), and/or CD127−, CD27+, CD57−, CD28− CD8 T cells, and/or CD127−, CD27−, CD57−, CD28− CD8 T cells, and/or CD127+, CD27−, CD57−, CD28− CD8 T cells, and/or associated CD8 T cell subsets shown in FIG. 10, and/or CCR7^{dim}, CD45RA+, CD8+ T cells in said blood sample;
providing a second blood sample from said subject at a second time after said first time;
performing the method according to any one of embodiments 1-13 to determine second levels of CD57+, CD28−, CD127−, CD27−, CD8+ T cells (SLEC CD8 T cells), and/or CD127−, CD27+, CD57−, CD28− CD8 T cells, and/or CD127−, CD27−, CD57−, CD28− CD8 T cells, and/or CD127+, CD27−, CD57−, CD28− CD8 T cells, and/or associated CD8 T cell subsets shown in FIG. 10, and/or CCR7^{dim}, CD45RA+, CD8+ T cells in said second blood sample, wherein:
an increase in the second levels of CD57+, CD28−, CD127−, CD27−, CD8+ T cells (SLEC CD8 T cells), and/or CD127−, CD27+, CD57−, CD28− CD8 T cells, and/or CD127−, CD27−, CD57−, CD28− CD8 T cells, and/or CD127+, CD27−, CD57−, CD28− CD8 T cells, and/or associated CD8 T cell subsets shown in FIG. 10 compared to the first level(s) of said T cells, and/or a decrease in CCR7^{dim}, CD45RA+, CD8+ T cells compared to the first levels of said T cells, indicates progression of said subject toward type 1 diabetes; and
no increase in the second levels of CD57+, CD28−, CD127−, CD27−, CD8+ T cells (SLEC CD8 T cells), and/or CD127−, CD27+, CD57−, CD28− CD8 T cells, and/or CD127−, CD27−, CD57−, CD28− CD8 T cells, and/or CD127+, CD27−, CD57−, CD28− CD8 T cells, and/or associated CD8 T cell subsets shown in FIG. 10 compared to the first level(s) of said T cells, and/or no decrease in CCR7^{dim}, CD45RA+, CD8+ T cells compared to the first levels of said T cells, indicates little or no progression of said subject toward type 1 diabetes.

Embodiment 54: The method of embodiment 53, wherein said method comprises determining first and second levels of CD57+, CD28−, CD127−, CD27−, CD8+ T cells (SLEC CD8 T cells).

Embodiment 55: The method according to any one of embodiments 53-54, wherein said method comprises determining first and second levels of CD127−, CD27+, CD57−, CD28− CD8 T cells.

Embodiment 56: The method according to any one of embodiments 53-55, wherein said method comprises determining first and second levels of CD127−, CD27−, CD57−, CD28− CD8 T cells.

Embodiment 57: The method according to any one of embodiments 53-56, wherein said method comprises determining first and second levels of CD127+, CD27−, CD57−, CD28− CD8 T cells.

Embodiment 58: The method according to any one of embodiments 53-57, wherein said method comprises determining first and second levels of or CCR7^{dim}, CD45RA+, CD8+ T cells in said blood sample.

Embodiment 59: The method according to any one of embodiments 53-58, wherein said increase in levels and/or said decrease in levels is a statistically significant increase or decrease.

Embodiment 60: The method of embodiment 59, wherein said increase in level(s) is a statistically significant increase or decrease in level(s) at a p value of p≤0.05, or p≤0.02, or p≤0.01.

Embodiment 61: The method according to any one of embodiments 53-60, wherein said subject has not progressed to clinical type 1 diabetes.

Embodiment 62: The method of embodiment 61, wherein said subject shows a normal hemoglobin A1c level ranging from 4% to 5.6%.

Embodiment 63: The method of embodiment 61, wherein said subject shows an elevated hemoglobin A1c level ranging from 5.7% to 6.4%.

Embodiment 64: The method of embodiment 61, wherein a random blood sugar test for said subject is below 200 mg/dL (11.1 mmol/L).

Embodiment 65: The method of embodiment 61, wherein a normal fasting blood sugar level for said subject is less than 100 mg/dL (5.6 mmol/L).

Embodiment 66: The method of embodiment 61, wherein an elevated fasting blood sugar level for said subject ranges from 100 to 125 mg/dL (5.6 to 6.9 mmol/L).

Embodiment 67: The method according to any one of embodiments 53-66, wherein said subject is a human and said blood or blood fraction is from said human.

Embodiment 68: A method of treating a subject to slow or prevent the onset of type 1 diabetes, said method comprising:
identifying a subject where the level of CD57+, CD28−, CD127−, CD27−, CD8+ T cells (SLEC CD8 T cells), and/or CD127−, CD27+, CD57−, CD28− CD8 T cells, and/or CD127−, CD27−, CD57−, CD28− CD8 T cells, and/or CD127+, CD27−, CD57−, CD28− CD8 T cells, and/or associated CD8 T cell subsets shown in FIG. 10 in blood or a blood fraction derived from said subject is elevated compared to the level(s) in normal healthy control, and/or or where the level of CCR7^{dim}, CD45RA+, CD8+ T cells in blood or a blood fraction from said subject is reduced compared to the level in a normal healthy control; and
treating said subject to slow or prevent the onset of type 1 diabetes.

Embodiment 69: The method of embodiment 68, wherein said method comprises identifying a subject where the level of CD57+, CD28−, CD127−, CD27−, CD8+ T cells (SLEC CD8 T cells) is elevated compared to the level in a normal healthy control and treating said subject to slow or prevent the onset of type 1 diabetes.

Embodiment 70: The method according to any one of embodiments 68-69, wherein said method comprises identifying a subject where the level of CD127−, CD27+, CD57−, CD28− CD8 T cells is elevated compared to the level in a normal healthy control and treating said subject to slow or prevent the onset of type 1 diabetes.

Embodiment 71: The method according to any one of embodiments 68-70, wherein said method comprises identifying a subject where the level of CD127−, CD27−, CD57−, CD28− CD8 T cells is elevated compared to the level in a normal healthy control and treating said subject to slow or prevent the onset of type 1 diabetes.

Embodiment 72: The method according to any one of embodiments 68-71, wherein said method comprises identifying a subject where the level of CD127+, CD27−, CD57−, CD28− CD8 T cells is elevated compared to the level in a normal healthy control and treating said subject to slow or prevent the onset of type 1 diabetes.

Embodiment 73: The method according to any one of embodiments 68-72, wherein said method comprises identifying a subject where the level of CCR7^{dim}, CD45RA+, CD8+ T cells is decreased compared to the level in a normal healthy control and treating said subject to slow or prevent the onset of type 1 diabetes.

Embodiment 74: The method according to any one of embodiments 68-73, wherein said treatment comprises administering an immune modulator to said subject.

Embodiment 75: The method of embodiment 74, wherein said immune modulator comprises an anti-viral agent, an anti-PD1 antibody, an anti-CTLA4 antibody, and/or an anti-CD3 antibody.

Embodiment 76: The method of embodiment 75, wherein said immune modulator comprises an anti-CD3 monoclonal antibody.

Embodiment 77: The method of embodiment 76, wherein said immune modulator comprises teplizumab.

Embodiment 78: The method of embodiment 75, wherein said immune modulator comprises an anti-viral agent.

Embodiment 79: The method of embodiment 78, wherein said immune modulator comprises an anti-viral agent selected from the group consisting of Pleconaril and Ribavirin.

Embodiment 80: The method according to any one of embodiments 68-79, wherein said method comprises re-induction of tolerance towards the putative self-antigen that causes T1D.

Embodiment 81: The method of embodiment 80, wherein said antigen comprises one or more antigen selected from the group consisting of insulin, glutamic acid decarboxylase (GAD), and the heat shock protein 60 (Hsp60)-derived peptide 277.

Embodiment 82: The method according to any one of embodiments 68-81, wherein said treating comprises monitoring blood glucose levels in said subject.

Embodiment 83: The method of embodiment 82, wherein said blood glucose is tested before meals and snacks, and/or before bed, and/or before exercising or driving.

Embodiment 84: The method of embodiment 82, wherein said blood glucose is monitored on a daily, a weekly, a biweekly, or a monthly basis.

Embodiment 85: The method according to any one of embodiments 68-84, wherein said treating comprises fitting said subject with a continuous glucose monitoring system.

Embodiment 86: The method according to any one of embodiments 68-85, wherein said treating comprises monitoring hemoglobin A1c.

Embodiment 87: The method according to any one of embodiments 68-86, wherein said treating comprise maintaining normal blood glucose levels.

Embodiment 88: The method of embodiment 87, wherein said maintaining normal blood glucose levels comprises administering insulin.

Embodiment 89: The method of embodiment 88, wherein said insulin comprises an insulin selected from the group consisting of short-acting (regular) insulin, rapid-acting insulin, intermediate-acting (NPH) insulin, and long-acting insulin.

Embodiment 90: The method according to any one of embodiments 68-89, wherein said treating comprises maintaining the subject on a low carbohydrate diet.

Embodiment 91: The method according to any one of embodiments 68-90, wherein said treating comprise maintaining said subject on a diet that provides a BMI of said subject ranging from about 18.5 to about 24.9.

Embodiment 92: The method according to any one of embodiments 68-91, wherein said treating comprise engaging said subject in daily exercise comprising at least 150 minutes of aerobic exercise a week, with no more than two days without any exercise.

Embodiment 93: The method according to any one of embodiments 68-92, wherein said treating comprises providing said subject enzyme (ACE) inhibitors or angiotensin II receptor blockers (ARBs).

Embodiment 94: The method according to any one of embodiments 68-93, wherein said treating comprises providing said subject aspirin.

Embodiment 95: The method according to any one of embodiments 68-94, wherein said treating comprises providing said subject cholesterol lowering drugs.

Embodiment 96: The method according to any one of embodiments 68-95, wherein said subject is a human.

Embodiment 97: A kit for the determination of risk for progression to type 1 diabetes, said kit comprising:
an anti-CD57 antibody, an anti-CD28 antibody, an anti-CD127 antibody, an anti-CD27 antibody, and an anti-CD8 antibody; and/or
an anti-CCR7 antibody, an anti-CD45RA antibody, and an anti-CD8 antibody.

Embodiment 98: The kit of embodiment 97, wherein said kit comprises an anti-CD57 antibody, an anti-CD28 antibody, an anti-CD127 antibody, an anti-CD27 antibody, and an anti-CD8 antibody.

Embodiment 99: The kit according to any one of embodiments 97-98, wherein said kit comprises an anti-CCR7 antibody, an anti-CD45RA antibody, and an anti-CD8 antibody.

Embodiment 100: The kit according to any one of embodiments 97-99, wherein said antibodies are each labeled with a detectable label.

Embodiment 101: The kit of embodiment 100, where the labels labeling each type of antibody are different and distinguishable.

Embodiment 102: The kit according to any one of embodiments 100-101, wherein said labels are fluorescent labels.

Embodiment 103: A method of determining if a subject is at risk for developing type 1 diabetes.

Embodiment 104: The method of embodiment 103, wherein the expression or lack of expression of at least one biomarker is determined, wherein said biomarker may include but is not limited to CD57, CD28, CD127, CD27, CD8, CD4, CD3, Va7.2. CD45RA, CCR7, CD161, CCR4, FOXP3, CD25, CXCR5, IgG, IgM, and IgA antibodies directed against CMV, CMV RNA, CMV DNA, and CMV proteins.

Embodiment 105: The method of embodiment 103, wherein specific T cell populations are identified and the presence of said T cell population or lack of said T cell populations are used to determine the relative risk for developing type 1 diabetes.

Embodiment 106: The method of embodiment 105, wherein the T cell populations may include but are not limited to CD8+, CD57+, CD28−, CD127−, CD27− T cells, CD3+, Va7.2+, CD161+, CD45RA−, CCR7−, CD28+, CD27−, CD8+/−, CD4+/− T cells, CD3+, Vα7.2+, CD161+, CD45RA^{low}, CCR7^{low}, CD28+, CD128+, CD8+/−, CD4+/− T cells, Vα7.2+. CD161+, CD45RA−, CCR7−, CD28+, CD27−, CD8+/−, CD4+/− T cells, CD4+, CD8−, CD127^{bright} CXCR5−, CCR4+, CCR7−, CD27+ T cells, CD8+, CD45RA+, CCR7^{dim}, CD57−, CD27+, CD127^{high}, CXCR5−, CCR4− T cells, CD8+, CD127−, CD27−, CD57+, CD28− T cells, CD4+, CD8+/−, CXCR5−, CCR4+, CCR7−, CD27+, CD127^{dim} T cells, CD4+, CD8+/−, CD127^{bright}, CD27+, CCR7−, CCR4+, CXCR5− T cells, CD4+, CCR4+, CXCR5+, CD161− T cells, CD4+, CD8−, CD27−, CCR7+, CCR4+, CXCR5− T cells, CD4+, CD8−, CD27−, CCR7−, CCR4+, CXCR5− T cells, CD4+, CD8−, CCR4+, CXCR5+, CD161− T cells, CD4+, CD8−, CD27−, CCR7+, CCR4+, CXCR5− T cells, CD4+, CD8−, CD27−, CCR7−, CCR4+, CXCR5− T cells, CD4+, CD8−, CCR4+, CXCR5+, CD161− T cells, or combinations thereof.

Embodiment 107: The method of embodiment 103, wherein the presence of the CMV and other Herpesvirus genome/DNA, CMV RNA, CMV proteins, or IgG, IgM, and IgA antibodies directed against CMV and other Herpesvirus are measured to identify increased risk of development of T1D.

Embodiment 108: The method of embodiment 107, wherein the CMV and other Herpesvirus markers are measured in conjunction with the T cell markers listed in embodiment 104 and/or embodiment 106 to determine a patient's risk of developing T1D.

Embodiment 109: A method of determining if a subject is at risk for developing type 1 diabetes, said method comprising:
a) determining the presence or absence of a specific T cell population in a sample obtained from said subject, wherein the T cell populations may include but are not limited to CD8+, CD57+, CD28−, CD127−, CD27− T cells, CD3+, Vα7.2+, CD161+, CD45RA−, CCR7−, CD28+, CD27−, CD8+/−, CD4+/− T cells, CD3+, Vα7.2+, CD161+, CD45RA^{low}, CCR7^{low}, CD28+, CD128+, CD8+/−, CD4+/− T cells, Vα7.2+, CD161+, CD45RA−, CCR7−, CD28+, CD27−, CD8+/−, CD4+/− T cells, CD4+, CD8−, CD127^{bright}, CXCR5−, CCR4+, CCR7−, CD27+ T cells, CD8+, CD45RA+, CCR7^{dim}, CD57−, CD27+, CD127^{high}, CXCR5−, CCR4− T cells, CD8+, CD127−, CD27−, CD57+, CD28− T cells, CD4+, CD8+/−, CXCR5−, CCR4+, CCR7−, CD27+, CD127^{dim} T cells, CD4+, CD8+/−, CD127^{bright}, CD27+, CCR7−, CCR4+, CXCR5− T cells, CD4+, CCR4+, CXCR5+, CD161− T cells, CD4+, CD8−, CD27−, CCR7+, CCR4+, CXCR5− T cells, CD4+, CD8−, CD27−, CCR7−, CCR4+, CXCR5− T cells, CD4+, CD8−, CCR4+, CXCR5+, CD161− T cells, CD4+, CD8−, CD27−, CCR7+, CCR4+, CXCR5− T cells, CD4+, CD8−, CD27−, CCR7−, CCR4+, CXCR5− T cells, CD4+, CD8−, CCR4+, CXCR5+, CD161− T cells, or combinations thereof, and
b) determining the presence or absence of CMV or other Herpesvirus DNA, CMV RNA, CMV proteins, or IgG, IgM, and IgA antibodies directed against CMV or other Herpesvirus in a sample obtained from said subject.

Embodiment 110: A method of preventing or treating type 1 diabetes in a subject, said method comprising:
a) determining the presence or absence of specific T cell populations in a sample obtained from said subject; and
b) determining the presence or absence of CMV or other Herpesvirus proteins, RNA, DNA, and/or IgG, IgA and IgM antibodies against CMV or other Herpesvirus, and
c) determining an appropriate therapy to prevent and/or treat type 1 diabetes based on the T cell population(s) that were identified; and
d) administering said therapy to said subject.

Embodiment 111: A composition or kit comprising at least one antibody for at least one biomarker selected from the group consisting of CD57, CD28, CD127, CD27, CD8, CD4, CD3, Vα7.2, CD45RA, CCR7, CD161, CCR4, FOXP3, CD25, CXCR5, CMV DNA, CMV or other Herpesvirus RNA, CMV or other Herpesvirus proteins, or IgG, IgA, and IgM antibodies to CMV or other Herpesvirus.

Embodiment 112: The composition or kit of embodiment 111, wherein said composition or kit also comprises a probe or antibody linked to a probe including but not limited to a fluorescent probe, radiolabeled probe, imaging agent, and/or contrast agent.

### Definitions

The terms “subject,” “individual,” and “patient” may be used interchangeably and refer to humans, as well as non-human mammals (e.g., non-human primates, canines, equines, felines, porcines, bovines, ungulates, lagomorphs, and the like). In various embodiments, the subject can be a human (e.g., adult male, adult female, adolescent male, adolescent female, male child, female child) under the care of a physician or other health worker in a hospital, as an outpatient, or other clinical context. In certain embodiments, the subject may not be under the care or prescription of a physician or other health worker.

The term “normal healthy control” when used with respect to the levels of various T cell populations (e.g., CD57+, CD28−, CD127−, CD27−, CD8+ T cells (SLEC CD8 T cells) and/or CCR7^{dim}, CD45RA+, CD8+ T cells) refers to the levels of the T cell populations in a typical subject that does not progress to type 1 diabetes. In certain embodiments the normal control level(s) refer to average levels determined for a population of subjects that does not progress to type 1 diabetes. In certain embodiments the “control” population can be matched to the test subject for gender, and/or for age, and/or for ethnicity.

In certain embodiments, the “level” of a T cell population can refer to the number of T cells in a particular sample, or can be normalized as, for example, the percentage and/or fraction of T cells in an analyzed sample.

“Type 1 diabetes mellitus” or “T1D”, once known as “juvenile diabetes” or “insulin-dependent diabetes (IDDM), is a chronic autoimmune disease associated with selective destruction of insulin-producing pancreatic β-cells. In addition to the loss of insulin secretion, the function of pancreatic α-cells is also abnormal and there is excessive secretion of glucagons in IDDM patients. Deficiency in insulin leads to uncontrolled lipolysis and elevated levels of free fatty acids in the plasma, which suppresses glucose metabolism in peripheral tissues such as skeletal muscle.

### BRIEF DESCRIPTION OF THE DRAWINGS

FIG. 1, panels A-D, shows that seroconverted subjects have reduced frequencies of MAIT cells. Panel A: Starting from total T cells, MAIT cells were identified as Vα7.2+, CD45RA−, CD161+, CCR7^{dim/-}, CD28+, CD127+ events. Panel B: Seroconverted subjects have reduced frequencies of MAIT cells in comparison to AA− subjects. Reduced frequencies were most acute in the CD8+, CD4− (CD8+) and CD8−, CD4− (DN) compartments (see, e.g., FIG. 11). Panel C: Upon dividing seroconverted subjects according to disease progression, we observed that reduced frequencies of MAIT cells were most prominent among non-progressors. Panel D: Descriptive statistics for the frequency of MAIT cells of total T cells for all groups compared. For panels B and C, bars represent median. Statistical tests fully described in Example 1 materials and methods.

FIG. 2, panels A-D, shows that seroconverted subjects have reduced frequencies of CD45RA+, CCR7^{dim} memory-like CD8 T cells. Panel A: Starting from total CD8 T cells. CD45RA+, CCR7^{dim} memory-like CD8 T cells were identified as CD28+, MAIT−, CCR4−, CXCR5−, CD127^{+/high}, CCR7^{dim}, CD27+, CD57- and CD45RA+. Panel B: Seroconverted subjects have reduced frequencies of CD45RA+, CCR7^{dim} memory-like CD8 T cells in comparison to AA− subjects. Reduced frequencies of CD45RA+, CCR7− memory-like CD8 T cells were also observed (FIG. 12). Panel C: Upon dividing seroconverted subjects according to disease progression, we observed that reduced frequencies of CD45RA+, CCR7^{dim} memory-like CD8 T cells were most prominent among non-progressors. Panel D: Descriptive statistics for the frequency of CD45RA+, CCR7^{dim} memory-like CD8 T cells of total T cells for all groups compared. For panels B and C, bars represent median. Statistical tests fully described in Example 1 materials and methods.

FIG. 3, panels A-D, shows that seroconverted subjects have elevated frequencies of short-lived effector-like cells (SLEC) and this expansion was most prominent among those that progressed to disease. Panel A: Starting from total CD8 T cells, SLEC were identified as CD28−, CD57+, CD27−, and CD127−. Panel B: Seroconverted subjects have increased frequencies of SLEC in comparison to AA− subjects. Panel C: Upon dividing seroconverted subjects according to disease progression, we observed that elevated frequencies of SLEC were most prominent among progressors. Panel D: Descriptive statistics for frequency of SLEC of total T cells for all groups compared. For panels B and C, bars represent median. Statistical tests fully described in Example 1 materials and methods.

FIG. 4, panels A-D, shows that seroconverted subjects have reduced frequencies of CCR4+, CD127^{bright} memory CD4 T cells. Panel A: Starting from total MAIT− CD4 T cells, CD127^{bright} memory T cells were identified as CCR4+, CXCR5−, CCR7−, CD27+, CD127^{bright} events. Panel B: Seroconverted subjects have reduced frequencies of CD127^{bright} memory CD4 T cells in comparison to AA− subjects. CCR4-expressing terminally differentiated and T follicular helper-like CD4 T cells were also reduced in frequency (FIG. 13). Panel C: Upon dividing seroconverted subjects according to disease progression, we observed that reduced frequencies of CD127^{bright} memory CD4 T cells were most prominent among non-progressors. Panel D: Descriptive statistics for the frequency of CD127^{bright} memory CD4 T cells of total T cells for all groups compared. For panels B and C, bars represent median. Statistical tests fully described in Example 1 materials and methods.

FIG. 5, panels A-D, shows that seroconverted subjects have reduced frequencies of CCR4+, CD127^{dim} Treg-like CD4 T cells. Panel A: Starting from total MAIT− CD4 T cells, CD127^{dim} Treg-like cells were identified as CCR4+, CXCR5−, CCR7−, CD27+, CD127^{dim} events. Panel B: Seroconverted subjects have reduced frequencies of CD127^{dim} Treg-like cells in comparison to AA− subjects. Panel C: Upon dividing seroconverted subjects according to disease progression, we observed that reduced frequencies of CD127^{dim} Treg-like CD4 T cells were most prominent among non-progressors. Panel D: Descriptive statistics for the frequency of CD127^{dim} Treg-like CD4 T cells of total T cells for all groups compared. For panels B and C, bars represent median. Statistical tests fully described in Example 1 materials and methods.

FIG. 6 shows that CCR4+, CXCR5− CD127^{dim} CD4 T cells possess a regulatory phenotype. A substantial population of FoxP3+, CD25^{high} events resides in the CXCR5−, CCR4+ subset. These FoxP3+ events generally exhibited low CD127 expression. Data is representative of 3 healthy subjects analyzed.

FIG. 7, panels A-F, shows that frequencies of CD127^{dim} Treg-like cells are positively correlated with CD127^{bright} memory CD4 T cells among seroconverted subjects. Panel A: We observed no significant correlation among frequencies of CD127^{dim} Treg-like cells and MAIT cells. Panel B: Although not correlated among AA− subjects, frequencies of CD127^{bright} memory and CD127^{dim} Treg-like CD4 T cells were positively and significantly correlated among seroconverted subjects. Panel C: Frequencies of CD45RA+, CCR7^{dim} memory-like CD8 T cells and CD127^{dim} Treg-like CD4 T cells were not correlated among seroconverted and AA− subjects. Panel D: Frequencies of SLEC were not correlated with CD127dim Treg-like CD4 T cells in AA- or seroconverted subjects. Panel E: Frequency of CD45RA+, CCR7dim memory-like CD8 T cells are positively correlated with islet cell antibody (ICA) levels. Panel F: Frequency of SLEC are positively correlated with harmonized insulinoma antigen 2 (IA-2H) levels. Statistical tests fully described in Example 1 materials and methods.

FIG. 8, panels A-H, shows that seroconverted subjects possessed elevated ratios of frequency of SLEC to both frequency of CD127^{dim} Treg-like CD4 cells and frequency of MAIT cells. The ratio of frequency of SLEC to frequency of CD Treg-like cells was significantly elevated among seroconverted subjects (panel A) and extended into both non-progressors and progressors (panel B). We observed no difference in ratios of either MAIT cells (panels C and D) or CD45RA+, CCR7^{dim} memory-like CD8 T cells (panels E and F) to CD127^{dim} Treg-like CD4 T cells. The ratio of frequency of SLEC to frequency of MAIT cells was significantly elevated among seroconverted subjects (panel G) and extended into both non-progressors and progressors (panel H). For all panels, bars represent median. Statistical tests fully described in Example 1 materials and methods.

FIG. 9, panels A-D, show that SLEC frequencies are strongly associated with cytomegalovirus IgG levels among seroconverted subjects, but not among controls. We gauged abundance of anti-viral immunoglobulins using ELISA. We then compared frequencies of T cell subsets with IgG abundance. Panel A: Frequency of MAIT cells was not significantly associated with abundance of cytomegalovirus (CMV) IgG, Epstein-Barr Virus viral capsid antigen (EBV VCA) IgG, or enterovirus IgG among seroconverted subjects and controls. Panel B: Frequency of CCR7^{dim}, CD45RA+CD8 T cells was significantly associated with EBV VCA IgG among seroconverted subjects, but not with CMV IgG or enterovirus IgG abundance. Panel C: Frequency of SLEC was significantly associated with CMV IgG abundance among seroconverted subjects but not among controls. We observed no significant relationship among SLEC with either EBV VCA IgG or enterovirus IgG. Panel D: CD127^{dim} Treg-like CD4 T cells were not significantly associated with CMV, EBV VCA, or enterovirus IgG abundance. Statistical tests fully described in Example 1 materials and methods.

FIG. 10, panels A-E, shows that SLEC and associated chronically-stimulated CD8 T cells are significantly elevated among CMV+ progressors. Subjects were stratified as either positive or negative for CMV IgG (CMV+ or CMV−). We then reanalyzed the CD28− CD8 T cell dataset to check for differences associated with CMV among the AA− non-progressor (Non-prog), and progressor (Prog) groups: Panel A: The SLEC expansion was most prominent among CMV+ progressors. We observed additional chronically-stimulated CD8 T cell subsets that were significantly expanded among the CMV+ progressor group. Panel B: CD127−, CD27+, CD57−, CD28−. Panel C: CD127−, CD27−, CD57−, CD28−. Panel D) CD127+, CD27−, CD57−, CD28−. The gating for the subsets in panels B, C, and D is shown in panel E. The CMV−, non-progressor that is colored grey and indicated with an arrow was equivocal for CMV IgG after being tested twice. Bars represent median. Statistical tests fully described in Example 1 materials and methods.

FIG. 11, panels A-B, shows that MAIT cell reductions were most acute among the CD8+(panel A) and DN (panel B) compartments. Gating is shown in FIG. 1, panel A. For panels A and B, bars represent median. Statistical tests fully described in Example 1 materials and methods.

FIG. 12, panels A-D, shows that seroconverted subjects have reduced frequencies of CD45RA+, CCR7− memory-like CD8 T cells. Panel A: Starting from total CD8 T cells, CD45RA+, CCR7− memory-like CD8 T cells were identified as CD28+, MAIT−, CCR4−, CXCR5−, CD127^{+/high}, CCR7−, CD27+, CD45RA+ events. Panel B: Seroconverted subjects have reduced frequencies of CD45RA+, CCR7− memory-like CD8 T cells in comparison to AA− subjects. Panel C: Upon dividing seroconverted subjects according to disease progression, we observed that reduced frequencies of CD45RA+, CCR7-memory-like CD8 T cells were most prominent among non-progressors. Panel D: Descriptive statistics for the frequency of CD45RA+, CCR7− memory-like CD8 T cells of total T cells for all groups compared. For panels B and C, bars represent median. Statistical tests fully described in Example 1 materials and methods.

FIG. 13, panels A-D, shows that seroconverted subjects have reduced frequencies of T follicular helper-like CD4 T cells. Panel A: Starting from total MAIT− CD4 T cells, T follicular helper-like CD4 T cells were identified as CXCR5+, CCR4+, CD161-events. Panel B: Seroconverted subjects have reduced frequencies of T follicular helper-like CD4 T cells in comparison to AA− subjects. Panel C: Upon dividing seroconverted subjects according to disease progression, we observed that reduced frequencies of T follicular helper-like CD4 T cells were most prominent among non-progressors. Panel D: Descriptive statistics for the frequency of T follicular helper-like CD4 T cells of total T cells for all groups compared. For panels B and C, bars represent median. Statistical tests fully described in Example 1 materials and methods.

FIG. 14 shows an interpretation of some of the T cell alterations described herein.

### DETAILED DESCRIPTION

In various embodiments novel biomarkers and assays using such biomarkers are provided for the detection, assessment of status, assessment of risk, prognosis, and diagnosis of Type 1Diabetes (T1D). Additionally, in certain embodiments, kits and methods for the detection, assessment of status, assessment of risk, prognosis, and diagnosis of T1D are provided. In particular, specific T cell markers and subpopulations of T cells have been identified as markers of T1D or progression to T1D and have utility in assessing a patient's probability of developing T1D. In certain embodiments markers for concurrent or previous cytomegalovirus (CMV) infection can be tested in addition to the T cell markers. These markers can evaluated in conjunction with viral markers to accurately predict disease development.

In various embodiments, biomarkers for presence and/or progression to T1D include the expression or lack of expression of specific cell surface markers on T cells. The cell surface markers include but are not limited to CD57, CD28, CD127, CD27, CD8, CD4, CD3, Vα7.2, CD45RA, CCR7, CD161, CCR4, FOXP3, CD25, and CXCR5. Specific sets or subpopulations of T cells were identified that expressed or lacked one or more of the cell surface markers that were associated with T1D. In one embodiment the T cell population is CD8+, CD57+, CD28−, CD127−, CD27−. In another embodiment the T cell population is CD3+, Vα7.2+, CD161+, CD45RA−, CCR7−, CD28+, CD27−, CD8+/−, CD4+/−. In another embodiment the T cell population is CD3+, Vα7.2+, CD161+, CD45RA−, CCR7−, CD28+, CD128+, CD8+/−, CD4+/−. In another embodiment the T cell population is Vα7.2+, CD161+, CD45RA−, CCR7−, CD28+, CD27−, CD8+/−, CD4+/−. In another embodiment the T cell population is CD4+, CD8−, CD127^{bright}, CXCR5−, CCR4+, CCR7−, CD27+. In another embodiment the T cell population is CD8+, CD45RA+, CCR7^{dim}, CD57−, CD27+, CD127^{high}, CXCR5−, CCR4−. In another embodiment the T cell population is CD8+, CD127−, CD27−, CD57+, CD28−. In another embodiment the T cell population is CD4+, CD8+/−, CXCR5−, CCR4+, CCR7−, CD27+, CD127^{dim}. In another embodiment the T cell population is CD4+, CD8+/−, CD127^{bright}, CD27+, CCR7−, CCR4+, CXCR5−. In another embodiment the T cell population is CD4+, CCR4+, CXCR5+, CD161−. In another embodiment the T cell population is CD4+, CD8−, CD27−, CCR7+, CCR4+, CXCR5−. In another embodiment the T cell population is CD4+, CD8−, CD27−, CCR7−, CCR4+, CXCR5−. In another embodiment the T cell population is CD4+, CD8−, CCR4+, CXCR5+, CD161−.

The T cell biomarkers and T cell populations were shown to increase and/or decrease in pre-diabetic patients and these changes correlated to their risk for developing Tl D. In one embodiment an increase in the CD8+, CD57+, CD28−, CD127−, CD27−, T cell population correlated with an increased risk of developing TID. Furthermore the CD8+, CD57+, CD28−, CD127−, CD27−, T cell population correlated to patients that were positive for CMV. Evaluation of this marker set in conjunction with measurement of IgG, IgM, and IgA antibodies directed against CMV allows prediction of development of T1D in subjects positive for the CMV virus. Patients with this biomarker may be candidates for specific treatments such as many different antivirals or immune suppressants. In another embodiment a decrease in Vα7.2+, CD161+, CD45RA−, CCR7−, CD28+, CD27−, CD8+/−, CD4+/− T cells was associated with an increased chance of developing autoantibodies and T1D. Patients with this biomarker may be candidates for T1D treatments including but not limited to anti-bacterials and anti-virals. In another embodiment a decrease in CD4+, CD8−, CD127^{bright}, CXCR5−, CCR4+, CCR7−, CD27+ T cells, CD4+, CD8−, CXCR5−, CCR4+, CCR7−, CD27+, CD127^{dim} T cells, CD4+, CD8−, CD27−, CCR7+, CCR4+, CXCR5− T cells, CD4+, CD8−, CD27−, CCR7−, CCR4+, CXCR5− T cells, or CD4+, CD8−, CCR4+, CXCR5+, CD161− T cells, or a combination thereof correlated with an increased risk of developing T1D. Patients with one or more of these biomarkers may be a candidate for TID treatments including but not limited to immune modulators, insulin, cell therapies (ex. T cell therapies), T cell growth factors, antivirals, and hormones.

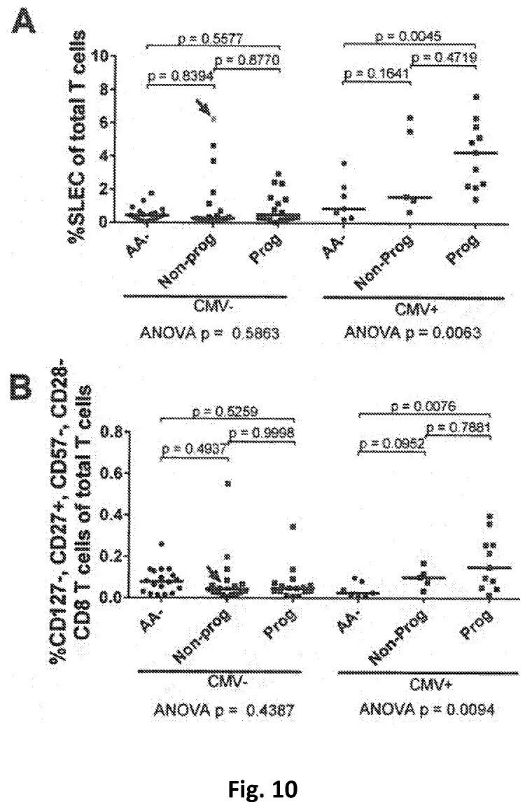

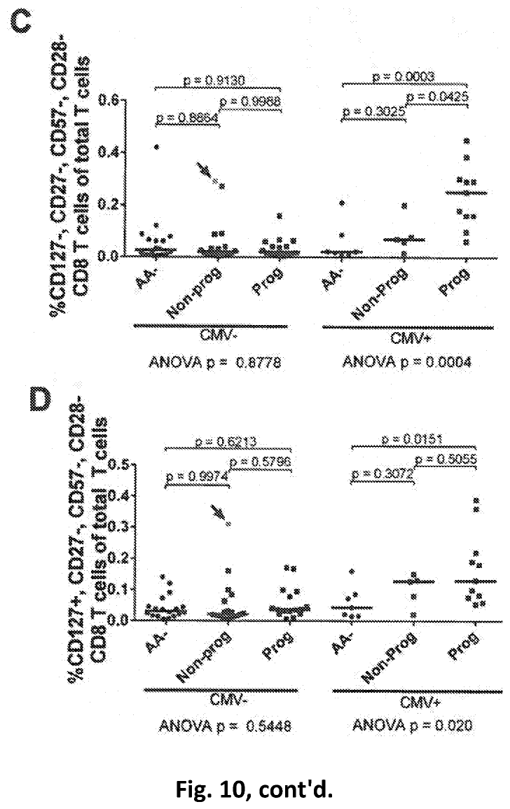

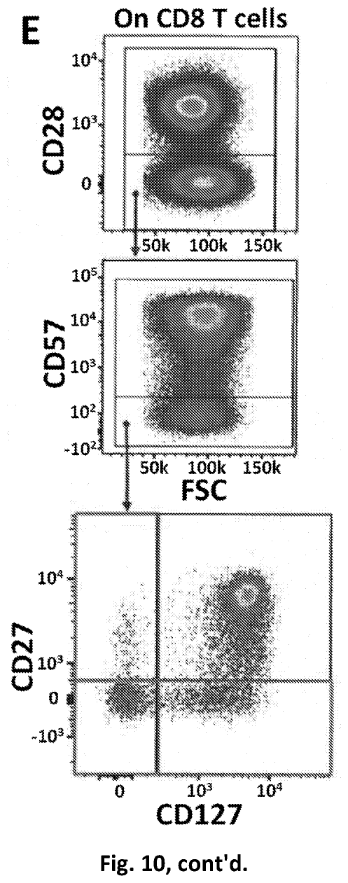

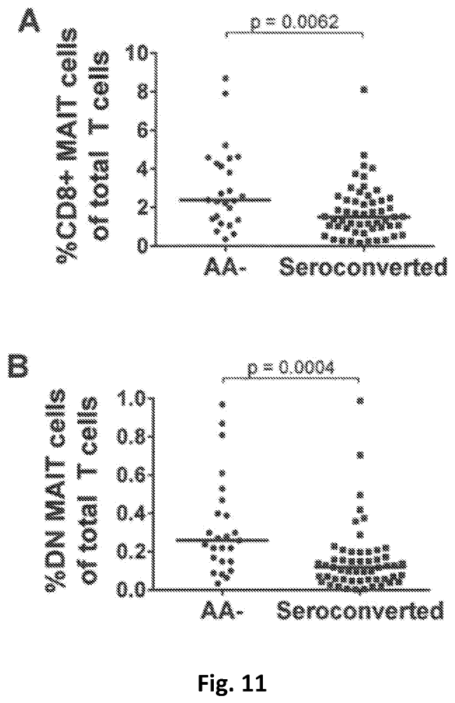

In particular, it was discovered, inter alia, that elevated levels of CD57+, CD28−, CD127−, CD27−, CD8+ T cells (SLEC CD8 T cells), and/or CD127−, CD27+, CD57−, CD28− CD8 T cells, and/or CD127−, CD27−, CD57−, CD28− CD8 T cells, and/or CD127+, CD27−, CD57−, CD28− CD8 T cells, and/or reduced levels of CCR7^{dim}, CD45RA+, CD8+ T cells are indicative of likely progression to type 1 diabetes (T1D). Accordingly, in certain embodiments, a method of determining if a subject is at risk for progression to type 1 diabetes is provided. In certain embodiments the method comprises determining the level(s) of CD57+, CD28−, CD127−, CD27−, CD8+ T cells (SLEC CD8 T cells), and/or CD127−, CD27+, CD57−, CD28− CD8 T cells, and/or CD127−, CD27−, CD57−, CD28− CD8 T cells, and/or CD127+, CD27−, CD57−, CD28− CD8 T cells, and/or associated CD8 T cell subsets shown in FIG. 10, and/or CCR7^{dim}, CD45RA+, CD8+ T cells in blood or a blood fraction derived from the subject, where: an elevated level of CD57+, CD28−, CD127−, CD27−, CD8+ T cells (SLEC CD8 T cells), and/or CD127−, CD27+, CD57−, CD28− CD8 T cells, and/or CD127−, CD27−, CD57−, CD28− CD8 T cells, and/or CD127+, CD27−, CD57−, CD28− CD8 T cells, and/or associated CD8 T cell subsets shown in FIG. 10 as compared to the level(s) of said T cells in a normal healthy control that does not progress to type 1 diabetes is an indicator that said test subject has a significant risk for progression to type 1 diabetes; and/or a reduced level CCR7^{dim}, CD45RA+, CD8+ T cells as compared to the level of said CCR7^{dim}, CD45RA+, CD8+ T cells T cells in a normal healthy control that does not progress to type 1 diabetes is an indicator that said test subject has a significant risk for progression to type 1 diabetes. In certain embodiments the method comprises determining the level of CD57+, CD28−, CD127−, CD27−, CD8+ T cells (SLEC CD8 T cells) in blood or a blood fraction derived from the subject, where an elevated level of SLEC CD8 T cells as compared to the level of SLEC CD8 T cells in a normal healthy control that does not progress to type 1 diabetes is an indicator that the subject has a significant risk for progression to type 1 diabetes. In certain embodiments the method comprises determining the level of CD127−, CD27+, CD57−, CD28− CD8 T cells in blood or a blood fraction derived from said test subject, where an elevated level of CD127−, CD27+, CD57−, CD28− CD8 T cells T cells as compared to the level of CD127−, CD27+, CD57−, CD28− CD8 T cells in a normal healthy control that does not progress to type 1 diabetes is an indicator that the subject has a significant risk for progression to type 1 diabetes. In certain embodiments the method comprises determining the level of CD127−, CD27−, CD57−, CD28− CD8 T cells in blood or a blood fraction derived from the subject, where an elevated level of said CD127−, CD27−, CD57−, CD28− CD8 T cells T cells as compared to the level of CD127−, CD27−, CD57−, CD28− CD8 T cells in a normal healthy control that does not progress to type 1 diabetes is an indicator that the subject has a significant risk for progression to type 1 diabetes. In certain embodiments the method comprises determining the level of CD127+, CD27−, CD57−, CD28− CD8 T cells in blood or a blood fraction derived from the subject, where an elevated level of said CD127+, CD27−, CD57−, CD28− CD8 T cells T cells as compared to the level of said CD127+, CD27−, CD57−, CD28− CD8 T cells in a normal healthy control that does not progress to type 1 diabetes is an indicator that the subject has a significant risk for progression to type 1 diabetes. In certain embodiments the method comprises determining the level of CCR7^{dim}, CD45RA+, CD8+ T cells in blood or a blood fraction from the subject, where a reduced level of said CCR7^{dim}, CD45RA+, CD8+ T cells as compared to the level of said T cells in a normal healthy control that does not progress to type 1 diabetes is a further indicator that the subject has a significant risk for progression to type 1 diabetes.

In certain embodiments a prognostic elevation or a prognostic decrease in the level of the above-identified T cell populations is a statistically significant elevated and/or reduced level compared to the normal healthy control. In certain embodiments the elevated and/or reduced level is a statistically significant elevated and/or reduced level compared to the normal healthy control(s) at a p value of p<0.05, or p≤0.02, or p≤0.01 (e.g., using Student's T test, and/or analysis of variance (ANOVA), and/or pairwise comparisons adjusting for multiple comparisons with Tukey's method where necessary).

In various embodiments the subject does not present with clinical type 1 diabetes (T1D), e.g., as determined by hemoglobin A1c (hbA1c), and/or a random blood sugar test, and/or a fasting blood sugar test. Accordingly, in certain embodiments the subject is asymptomatic, e.g., with a normal hemoglobin A1c (HbA1c) level ranging from 4% to 5.6%, and/or a normal fasting blood sugar level less than 100 mg/dL (5.6 mmol/L).

In certain embodiments the subject presents as pre-diabetic, e.g., as determined by hemoglobin A1c (hbA1c), and/or a random blood sugar test, and/or a fasting blood sugar test. Accordingly, in certain embodiments, the subject shows an elevated hemoglobin A1c (HbA1c) level ranging from 5.7% to 6.4%; and/or an elevated fasting blood sugar level for the subject ranges from 100 to 125 mg/dL (5.6 to 6.9 mmol/L).

In certain embodiments the subject is a subject that has been identified as seroconverted for autoantibodies. In certain embodiments such identification can be made by screening blood or a blood fraction from the subject for antibodies directed against one of more biomarkers selected from the group consisting of GAD65, IA-2, and IAA. In certain embodiments after a positive identification of antibodies against one or more of the GAD65, IA-2, and/or IAA biomarkers, blood or a blood fraction derived from the subject is screened for the biomarkers ZnT8 and/or ICA. In certain embodiments positive thresholds for the biomarkers are: IAA>0.010, GAD65>0.032, ICA512>0.049, GAD65H>20; IA-2H>5, ZnT8>0.020 and/or ICA≥10.

In certain embodiments, particularly, where the subject tests positive for elevated levels of the T cell populations identified above, the subject may be evaluated for the presence of an active viral infection as a further indicator of possible progression to T1D. In certain embodiments the subject is evaluated for an active viral infection by screening for viral IgM positivity, and/or analyzing plasma or circulating leukocytes for presence and abundance of viral nucleic acids. In certain embodiments the viral infection is a CMV or other Herpesvirus infection.

In various embodiments, the results of the assays described herein, or a diagnosis/prognosis based thereon can be recorded in any of a variety of media (e.g., in a medical record). In certain embodiments, the medical record can be maintained by a laboratory, physician's office, a hospital, a health maintenance organization, an insurance company, or a personal medical record website. In certain embodiments, the results of the assays described herein, or a diagnosis/prognosis based thereon can be recorded on or in a medic alert article selected from the group consisting of a card, worn article, or radiofrequency identification (RFID) tag, and the like. In certain embodiments, the results of the assays described herein, or a diagnosis/prognosis based thereon is recorded on a non-transient computer readable medium.

It will be recognized that the assays described herein themselves need not be dispositive of the presence or prognosis of progression to T1D. Typically such an evaluation will be made in the context of a differential diagnosis that considers other factors (e.g., hbA1c, blood glucose, body weight, lifestyle, etc.). In certain embodiments, particularly where the subject presents as pre-diabetic, the assays provided here are useful to further evaluate the likelihood of progression to T1D and can inform the level of intervention to be undertaken.

In certain embodiments, the assays described herein can also be used to evaluate the progression of a subject to type 1 diabetes. Thus, in one illustrative, but non-limiting example, a method of monitoring the onset and/or progression of type 1 diabetes in a subject is provided. In certain embodiments the method involves providing a blood sample from the subject at a first time, performing the assay methods described herein (e.g., to determine first/initial levels of CD57+, CD28−, CD127−, CD27−, CD8+ T cells (SLEC CD8 T cells), and/or CD127−, CD27+, CD57−, CD28− CD8 T cells, and/or CD127−, CD27−, CD57−, CD28− CD8 T cells, and/or CD127+, CD27−, CD57−, CD28− CD8 T cells, and/or associated CD8 T cell subsets shown in FIG. 10, and/or CCR7^{dim}, CD45RA+, CD8+ T cells); providing a second blood sample from the subject at a second time after the first time; again performing the assay methods described herein (e.g., to determine levels of CD57+, CD28−, CD127−, CD27−, CD8+ T cells (SLEC CD8 T cells), and/or CD127−, CD27+, CD57−, CD28− CD8 T cells, and/or CD127−, CD27−, CD57−, CD28− CD8 T cells, and/or CD127+, CD27−, CD57−, CD28− CD8 T cells, and/or associated CD8 T cell subsets shown in FIG. 10, and/or CCR7^{dim}, CD45RA+, CD8+ T cells) where an increase in the second levels of CD57+, CD28−, CD127−, CD27−, CD8+ T cells (SLEC CD8 T cells), and/or CD127−, CD27+, CD57−, CD28− CD8 T cells, and/or CD127−, CD27−, CD57−, CD28− CD8 T cells, and/or CD127+, CD27−, CD57−, CD28− CD8 T cells, and/or associated CD8 T cell subsets shown in FIG. 10 compared to the first level(s) of said T cells, and/or a decrease in CCR7^{dim}, CD45RA+, CD8+ T cells compared to the first level(s) of said T cells, indicates progression of said subject toward type 1 diabetes; and no increase in the second levels of CD57+, CD28−, CD127−, CD27−, CD8+ T cells (SLEC CD8 T cells), and/or CD127−, CD27+, CD57−, CD28− CD8 T cells, and/or CD127−, CD27−, CD57−, CD28− CD8 T cells, and/or CD127+, CD27−, CD57−, CD28− CD8 T cells, and/or associated CD8 T cell subsets shown in FIG. 10 compared to the first level(s) of said T cells, and/or no decrease in CCR7^{dim}, CD45RA+, CD8+ T cells compared to the first levels of said T cells, indicates little or no progression of said subject toward type 1 diabetes.

In certain embodiments, to indicate progression towards T1D, the increase and/or decrease in levels is a statistically significant increase and/or decrease of second level(s) as compared to the first levels. In certain embodiments the increase and/or decrease in second level(s) of the indicated T cell population(s) is a statistically significant elevated and/or reduced level compared to the first level(s) of the indicated T cell population(s) at a p value of p≤0.05, or p≤0.02, or p≤0.01 (e.g., using Student's T test, and/or analysis of variance (ANOVA), and/or pairwise comparisons adjusting for multiple comparisons with Tukey's method where necessary). In certain embodiments where progression towards T1D is observed, increased intervention is warranted.

It will be recognized, that in various embodiments, the assays described herein are performed on “fresh” blood samples or fractions derived therefrom. However, it will also be recognized that the assays can be performed on frozen or cryopreserved blood samples.

In certain embodiments, where the difference in first and second level(s) of the above-identified T cell populations indicates progression toward or to T1D, treatment/intervention may be undertaken or where already underway, pursued more aggressively (e.g., as described below).

### Identification/Quantification of Biomarkers and T Cell Populations.

Illustrative methods for quantifying the biomarkers and T cell populations include but are not limited to flow cytometry, mass cytometry or CYTOF, microfluidic cell sorting, ELISA measurements, western blotting, methods to detect viral DNA such as PCR, and methods to detect viral proteins such as immunohistochemistry and western blotting.

In certain embodiments methods of quantifying the T cell populations relevant to the assays described herein (e.g., quantifying CD57+, CD28−, CD127−, CD27−, CD8+ T cells (SLEC CD8 T cells), and/or CCR7^{dim}, CD45RA+, CD8+ T cells)) involve providing a biological sample from the subject containing T cells and labeling the cells with labels that are specific for the markers of interest, e.g., CD57, CD28, CD127, CD27, CCR7, CD45RA, CD8, etc. The cells can then be screened for the presence or absence of these markers thereby identifying the T cell subpopulation.

Antibodies specific to these cell surface markers are well known to those of skill in the art and are commercially available. Thus, as illustrated in Example 1, suitable antibodies and reagents include, but are not limited to: from Biolegend CCR7 (PE, clone G043H7), CCR4 (PE-Cy7, clone L291H4), CD14 (BV711, clone M5E2), CD19 (BV711, clone HIB19), CD27 (BV785, clone 0323), CD57 (F1TC, clone HCD57), CD45RA (PerCP-Cy5.5, clone HI100), Vα7.2 (BV605, clone 3C10), CD45 (AF700, clone HI30), Streptavidin-APC; from BD Biosciences CXCR5 (BV421, clone RF8B2), CD3 (BUV737, clone UCHT1), CD4 (BUV395, clone RPA-T4), CD8 (BV510, clone RPA-T8), CD28 (PE-CF594, clone CD28.2), CD161 (BV650. clone DX12); from eBioscience CD127 (Biotin, clone eBioRDR5); from life technologies LiveDead UV Blue.

In various embodiments, as indicated above, the antibodies are labeled. In certain embodiments the antibodies are labeled with different and distinguishable labels to facilitate identification of the different cell-surface markers.

As noted above, methods of screening the cells are well known to those of skill in the art and are exemplified in Example 1. In one illustrative, but non-limiting embodiments, the cells are screened using flow cytometry. One illustrative flow cytometer is the BD LSR II is a flow cytometer offered by BD Biosciences. This instrument provides an advanced fluidics system, high-performance data acquisition and analysis, and a unique optics system. The BD LSR II is designed with 4 lasers line colors (488 nm blue, 640 nm red, 405 nm violet, and 355 nm UV). The system is capable of detecting up to 12 parameters at the same time. The laser power for the excitation optics varies. The blue 488 nm laser's power is 20 mW. A laser power of 40 mW applies to the Red 640 nm laser. The UV 355 nm laser has laser power of 20 mW. Finally, the Violet laser with a wavelength of 405 nm has a laser power of 25 mW. This flow cytometer is illustrative and non-limiting and one of skill will recognize that numerous other instruments can be used to perform the analyses described herein.

### Methods of Treatment/Intervention.

As indicated above, where the assays described herein provide an increased likelihood of progression to type 1 diabetes (T1D) and/or where the monitoring methods described herein show actual progression toward T1D, the subject may be treated therapeutically or prophylactically to slow or prevent the onset of T1D, or to mitigate the symptoms or progression of present T1D. Illustrative methods of treatment/intervention include, but are not limited to immune modulators (e.g., anti-viral agents (e.g., Pleconaril, Ribavirin, and the lie), anti-PD1 antibodies, anti-CTLA4 antibodies, anti-CD3 antibodies, and the like), insulin, cell therapies (ex. T cell therapies), T cell growth factors, antivirals, and hormones.

In certain embodiments prevention or delay of the onset of type 1 diabetes mellitus can involve early intervention in the autoimmune process directed against β cells of the pancreatic islets of Langerhans. This autoimmune inflammatory process is thought to be caused by the effect of T cells and their secreted cytokines (e.g., interferon) and to be suppressed by regulatory T cells. Various methods aimed specifically at halting or modulating this response have been attempted. An alternative method is the re-induction of tolerance towards the putative self-antigen that causes the disease. Illustrative antigens such as insulin, glutamic acid decarboxylase (GAD) and the heat shock protein 60 (Hsp60)-derived peptide 277 have been used successfully in murine diabetes models and in initial clinical trials in early diabetes patients.

Additionally, researchers have demonstrated that immune therapy can slow the onset of type 1 diabetes in individuals at risk for this chronic condition. A 2-week regimen of teplizumab, an experimental anti-CD3 monoclonal antibody that interferes with the body's autoimmune destruction of insulin-producing cells, slowed development of the disease by nearly 2 years in high risk patients (see, e.g., Herold et al. (2019) N. Engl. J. Med. 381: 603-613).

Typically treatment for type 1 diabetes includes, but need not be limited to: 1) Taking insulin, 2) Carbohydrate, fat and protein counting, 3) Blood sugar monitoring, 4) Healthy diet, and 5) Exercising and maintaining a healthy weight. Typically, the goal is to maintain blood sugar and HbA1c as close to normal. In certain embodiments the goal is to keep daytime blood sugar levels before meals between about 80 and about 130 mg/dL (4.44 to 7.2 mmol/L) and after-meal numbers no higher than 180 mg/dL (10 mmol/L) two hours after eating.

In certain embodiments the treatment/intervention involves monitoring blood sugar levels. In certain embodiments blood sugar is tested before meals and snacks, and/or before bed, and/or before exercising or driving. In certain embodiments blood sugar is measured 4 times/day, or 2 times/day, or daily, or weekly, or biweekly, or on a monthly basis. In certain embodiments blood sugar is monitored by fitting the subject with a continuous glucose monitoring system. In certain embodiments treatment involves monitoring hemoglobin A1c, e.g., on a daily, weekly, biweekly, monthly, every 2 months, every 3 months, every 6 months, or on a yearly basis.

In certain embodiments the subject can be administered insulin. Illustrative types of insulin include, but are not limited to short-acting (regular) insulin, rapid-acting insulin, intermediate-acting (NPH) insulin, and long-acting insulin. Examples of short-acting (regular) insulin include HUMULIN R® and NOVOLIN R®. Rapid-acting insulin examples are insulin glulisine (APIDRA®), insulin lispro (HUMALOG®) and insulin aspart (NOVOLOG®). Long-acting insulins include insulin glargine (LANTUS®, TOUJEO SOLOSTAR®), insulin detemir (LEVEMIR®) and insulin degludec (TRESIBA®). Intermediate-acting insulins include insulin NPH (NOVOLIN N®, HUMULIN N®).

In certain embodiments the subject can be provided with insulin via injection or using an insulin pump. In certain embodiments the subject can be fitted with an Artificial pancreas. In September 2016, the Food and Drug Administration approved the first artificial pancreas for people with type 1 diabetes who are age 14 and older. It's also called closed-loop insulin delivery. The implanted device links a continuous glucose monitor, which checks blood sugar levels every five minutes, to an insulin pump. The device automatically delivers the correct amount of insulin when the monitor indicates it's needed.

In certain embodiments additional medications for subjects at risk for or with type 1 diabetes, such as may be administered. These include, but are not limited to: 1) High blood pressure medications such as angiotensin-converting enzyme (ACE) inhibitors or angiotensin II receptor blockers (ARBs) to maintain kidney health, to help keep your kidneys healthy, particularly for subjects with blood pressures above 140/90 millimeters of mercury (mm Hg); 2) Aspirin for cardiac protection; and 3) Cholesterol-lowering drugs e.g., statins (HMG-CoA reductase inhibitors) such as atorvastatin (LIPITOR®), fluvastatin (LESCOL®, LESCOL XL®), lovastatin (MEVACOR®, ALTOPREV®), pravastatin (PRAVACHOL®), rosuvastatin (CRESTOR®), simvastatin (ZOCOR®), pitavastatin (LIVALO®), and the like.

In certain embodiments intervention to slow or prevent the onset or progression of T1D includes regulation of diet and monitoring of carbohydrates. In this regard it is recommended to center the diet on nutritious, low-fat, high-fiber foods such as fruits, vegetables, whole grains, and the like. In certain embodiments carbohydrates and other high glycemic index foods are significantly reduced.

In certain embodiments physical activity is recommended. In certain embodiments the wherein said treatment comprise engaging the subject in daily exercise comprising at least 150 minutes of aerobic exercise a week, with no more than two days without any exercise.

In certain embodiments the treatment/intervention comprises heightened monitoring for symptoms of low blood sugar. Such symptoms include, but are not limited to sweating, shakiness, hunger, dizziness or lightheadedness, rapid or irregular heart rate, fatigue, headaches, blurred vision, and irritability. Other symptoms of low blood sugar include, but are not limited to lethargy, confusion, behavior changes, sometimes dramatic, poor coordination, and convulsions. In certain embodiments the treatment/intervention comprises heightened monitoring for hyperglycemia. Such symptoms include, but are not limited to frequent urination, increased thirst, blurred vision, fatigue, irritability, hunger, and difficulty concentrating.

Additionally, in certain embodiments, the treatment/intervention comprises increased monitoring for increased ketones in urine (diabetic ketoacidosis) of the subject. Illustrative symptoms of ketoacidosis include, but are not limited to nausea, vomiting, abdominal pain, a sweet, fruity smell to the breath, and weight loss.

The foregoing methods of treatment/intervention are illustrative and non-limiting.

### Kits.

In certain embodiments, kits for detecting the biomarkers including the specific T cell populations and CMV viral antigen are also envisioned as part of this invention. Kit components may include but are not limited to antibodies (monoclonal or polyclonal) to cell surface markers such as CD57, CD28, CD127, CD27, CD8, CD4, CD3, V0172, CD45RA, CCR7, CD161, CCR4, and CXCR5, and live/dead discriminating dyes. Antibodies to CMV and reagents to detect IgG, IgM, and IgA antibodies against CMV may also be included in the kits. Kits may also include compounds or probes that may aid in the detection of the biomarkers such as fluorescent probes, radiolabeled probe, imaging agents, contrast agents, antibodies labeled with fluorescent probes, blocking reagents, isotype antibodies, biotinylated antibodies, antibodies with conjugated dyes, antibody coated microwell plates, labeled avidin/dye complexes, CMV proteins, and buffers.

In certain embodiments the kits comprise an anti-CD57 antibody, an anti-CD28 antibody, an anti-CD127 antibody, and an anti-CD8 antibody; and/or an anti-CCR7 antibody, an anti-CD45RA antibody, and an anti-CD8 antibody. In certain embodiments the kits comprise an anti-CD57 antibody, an anti-CD28 antibody, an anti-CD127 antibody, and an anti-CD8 antibody. In certain embodiments the kits comprise an anti-CD127 antibody, an anti-CD57 antibody, an anti-CD28 antibody, an anti-CD27 antibody, and an anti-CD8 antibody. In certain embodiments the kits comprise an anti-CCR7 antibody, an anti CD45RA antibody, and an anti-CD8 antibody.

In certain embodiments the antibodies are each labeled with a detectable label. In certain embodiments the labels labeling each type of antibody are different and distinguishable. In certain embodiments the labels are fluorescent labels.

Additionally, in certain embodiments, the kits can include instructional materials disclosing protocols for the use of the reagents contained therein (e.g., antibodies) for evaluating the levels of T cell populations (e.g., CD57+, CD28−, CD127−, CD27−, CD8+ T cells (SLEC CD8 T cells) and/or CCR7^{dim}, CD45RA+, CD8+ T cells) that are markers for progression to T1D.

While the instructional materials in the various kits typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

### EXAMPLES

The following examples are offered to illustrate, but not to limit the claimed invention.

### Example 1

### Abnormal T Cell Frequencies Including Cytomegalovirus-Associated Expansions Distinguish Seroconverted Subjects at Risk for Type I Diabetes

As described in this example, we analyzed T cell subsets front cryopreserved PMBC obtained from the TrialNet Pathway to Prevention archives. We compared subjects who had previously seroconverted for one or more autoantibodies with non-seroconverted, autoantibody negative individuals. We observed a reduced frequency of MAIT cells among seroconverted subjects. Seroconverted subjects also possessed decreased frequencies of CCR4−-expressing CD4 T cells, including a regulatory-like subset. Interestingly, we found an elevation of CD57+, CD28−, CD127−, CD27−, CD8 T cells (SLEC) among seroconverted subjects that was most pronounced among those that progressed to disease. The frequency of these SLEC was strongly correlated with CMV IgG abundance among seroconverted subjects, associated with IA-2 levels, and most elevated among CMV+ seroconverted subjects who progressed to disease. Combined, our data indicate discrete, yet profound T cell alterations are associated with islet autoimmunity among at-risk subjects.

### Materials and Methods.

Patient Samples

Cryopreserved PBMC were acquired as part of an ancillary study approved by TrialNet from the Pathway to Prevention study of 1st and 2nd degree relatives of individuals with type 1 diabetes. Aside from the familial association, study eligibility is limited to those 1st and 2nd degree relatives who are not on medication to control hyperglycemia, not currently using immunosuppressive or immunomodulatory agents have no known severe active disease, and do not have diabetes or a history of diabetes. From this pool of subjects we received 79 blinded samples comprising 3 subgroups of subjects. Twenty-five samples were from autoantibody negative (AA−) 1st and 2nd degree relatives of diagnosed type 1 diabetics or seroconverted at-risk individuals having no autoantibodies, yet sharing some environmental and genetic risk. These samples constitute our control group. Importantly, these AA− subjects were not related to the seroconverted, at-risk individuals analyzed in our study. The remaining 54 subjects, labeled “seroconverted”, had previously seroconverted for one or more autoantibodies and are at increased risk of developing T1D (Bingley et al. (2016) Diabetologia, 59: 542-549; Vehik et al. (2011) Diabetes Care, 34: 1897-1901). Autoantibody testing was performed by TrialNet. Individuals were screened for GAD65, IA-2, and IAA. If any of these measurements were positive, they were also then screened for ZnT8 and ICA. Positivity thresholds were as follows: IAA, >0.010; GAD65, >0.032; ICA512, >0.049; GAD65H; >20; IA-2H, >5; ZnT8, >0.020; ICA, ≥10. Once an individual has tested positive for an autoantibody twice within one year, they are considered “autoantibody positive” or seroconverted to the production of autoantibodies targeting islet antigens.

Among the seroconverted subset, 26 samples were from individuals who have not yet progressed to clinical disease, and for our analysis these are labeled “non-progressor”. The remaining 28 samples were from individuals who ultimately progressed to clinical disease. This subgroup is labeled as “progressor”. Our request for samples from TrialNet did not specify particular HLA-risk alleles. Therefore our sample set constituted a mix of HLA types. Although we did request samples from subjects between 8-11 years old, upon unblinding the samples we discovered our subjects spanned a much broader age range. Patient data is presented in Table 1.

TABLE 1
Patient data.

| | | N (n | Vari- | | | Medi- | | |
|---|---|---|---|---|---|---|---|---|
| Status | | female) | able | Mean | SD | an | Min | Max |
| AA- | | 25(11) | Age | 12.2 | 2.7 | 12.0 | 7.7 | 16.9 |
| | | | BMI | 19.7 | 5.4 | 18.7 | 14.5 | 35.6 |
| | | | HbA1c | 5.1 | 0.3 | 5.1 | 4.5 | 5.6 |
| Sero- | | 54(23) | Age | 10.9 | 4.6 | 10.3 | 4.7 | 26.8 |
| converted | | | BMI | 20.4 | 5.5 | 19.1 | 14.0 | 45.7 |
| | | | HbA1c | 5.1 | 0.3 | 5.1 | 4.3 | 6.0 |
| Sero- | Non- | 26(10) | Age | 10.6 | 4.0 | 9.9 | 4.8 | 21.8 |
| converted | prog- | | BMI | 19.8 | 4.6 | 18.2 | 14.0 | 29.8 |
| | ressor | | HbA1c | 5.0 | 0.3 | 4.9 | 4.5 | 5.8 |
| | Prog- | 28(13) | Age | 11.3 | 5.1 | 10.8 | 4.7 | 26.8 |
| | ressor | | BMI | 21.1 | 6.2 | 20.7 | 14.8 | 45.7 |
| | | | HbA1c | 5.2 | 0.4 | 5.3 | 4.3 | 6.0 |

Flow Cytometry

Surface panel design and analysis was performed using a BD LSR II equipped with 5 lasers: UV (355 nm) for Brilliant Ultraviolet (BUV) 395 and 737, and LiveDead UV Blue; Violet (405 nm) for Brilliant Violet (BV) 421, 510, 605, 650, 711 and 785; Blue (488 nm) for FITC and PerCP-Cy5.5; Yellow-Green (561 nm) for PE, PE-CF594. and PE-Cy7; and Red (633 nm) for APC and Alexa Fluor (AF) 700. The following antibodies and reagents were used: from Biolegend CCR7 (PE, clone G043H7), CCR4 (PE-Cy7, clone L291H4), CD14 (BV711, clone M5E2), CD19 (BV711, clone H1B19), CD27 (BV785, clone 0323), CD57 (F1TC, clone HCD57), CD45RA (PerCP-Cy5.5, clone HI100), Vα7.2 (BV605, clone 3C10), CD45 (AF700, clone HI30), Streptavidin-APC; from BD Biosciences CXCR5 (BV42, clone RF8B2), CD3 (BUV737, clone UCHT1), CD4 (BUV395, clone RPA-T4), CD8 (BV510, clone RPA-T8), CD28 (PE-CF594, clone CD28.2), CD161 (BV650. clone DX12); from eBioscience CD127 (Biotin, clone eBioRDR5); from life technologies LiveDead UV Blue. Cryopreserved PBMC were thawed and rested overnight in X-VIVO 15 (Lonza) supplemented with human AB serum (MP Biomedicals) at 2% v/v. Samples were then counted and distributed to 96 well plates for labeling following methods described previously (Harms et al. (2017) Frontiers Immunol. 8: 1020) with two modifications: Fc receptors were blocked using human IgG (Sigma Aldrich) and antibody cocktails were prepared using Brilliant Stain Buffer (BD Biosciences).

For FoxP3 targeting healthy human lymphocytes were acquired from the UNMC elutriation core facility, cryopreserved, thawed, rested and surface labeled as described above. We then utilized the True-Nuclear Transcription Factor Buffer set (BioLegend) according to manufacturer's recommendations. In panel validation we compared PIE-conjugated FoxP3 clones 206D (BioLegend), PCH 01 (eBioscience), and 259D/C7 (BD Biosciences) and clone 206D gave the greatest signal-to-noise ratio (data not shown). The final panel included the aforementioned FoxP3, CD25 (VioBright FITC, clone 4E3, Miltenyi), and the conjugated-clones and reagents listed above for LiveDead, CD3, CD4, CXCR5 CCR4, and CD127 with streptavidin-APC.

Data analysis was conducted in a blinded fashion using FlowJo (v10.2. TreeStar). All samples were pregated to exclude doublets, debris, dead cells, and non T cells (lineage negative) and then multiple subsets were identified and gated according to known T cell differentiation pathways. Following analysis, raw data files and tabular results were uploaded to TrialNet, upon which we received sample identification and clinical data in order to perform statistical analysis.

Statistics

For mean comparisons, natural log transformation was applied to the data prior to analysis. For comparison of two groups, we used Student's T test and for comparisons of three groups, we used analysis of variance (ANOVA). If the results of the ANOVA were significant, then pairwise comparisons were made, adjusting for multiple comparisons with Tukey's method. For correlations, data were not transformed and we used Spearman's Rank-Order correlation test. In all cases, figures and descriptive data depict non-transformed data to facilitate interpretation. Figures were made using Prism (v6.03 GraphPad Software Inc), and data was analyzed using Prism and R.

### Results

Reduced Frequencies of Mucosal Associated Invariant T Cells (MAIT) in Seroconverted Subjects

MAIT cells are regularly found in human blood, the liver, and the gastrointestinal mucosa (Dusseaux et al. (2011) Blood, 117: 1250-1259), and respond to microbially-derived vitamin B metabolites presented through MR1 (Eckle et al. (2015) J Biol. Chem. 290: 30204-30211). MAIT cells are capable of directly lysing bacterially-sensitized and infected target cells (Kurioka et al. (2015) Mucosal Immunol. 8: 429-440; Le Bourhis et al. (2013) PLos Pathogens, 9: e1003681) and can produce the proinflammatory cytokines IFN-γ, TNF-α, and IL-17 (Dusseaux et al. (2011) Blood, 117: 1250-1259). Phenotypically, in the absence of MRI-loaded multimers, MAIT cells are unambiguously identifiable in blood by the high CD161 expression, presence of Vα7.2 T cell receptor, and T cell lineage marker CD3 (Chen et al. (2017) Mol. Metab. 6: 943-2957; Reantragoon et al. (2013) J Exp. Med. 210(11): 2305-2320). The circulating MAIT cell lineage in healthy subjects is also regularly CD28+, CD127+, CD45RA^{low}, and CCR7^{low} (Dusseaux et al. (2011) Blood, 117: 1250-1259; Dias et al. (2017) Proc. Natl. Acad. Sci. USA, 114: E5434-E5443), but exceptions to this phenotype do exist in certain disease states (e.g., chronic HIV (Leeansyah et al. (2013) Blood, 121: 1124-1135)), as well as in fetal tissue and cord blood (Leeansyah et al. (2014) Nat. Comm. 5: 3143; Martin et al. (2009) PLoS Biol. 7: e1000054). MAIT cells are generally CD8+(˜85%) with the remaining being chiefly DN (CD4−, CD8-), although CD4+ and DP (CD4+, CD8+) groups are frequently observable in scant amounts in healthy donors. The functional differences among these MAIT cell subsets have not been fully clarified, although it has recently been reported that the CD8+ subset possesses somewhat greater effector potential than the DN subset (Brozovz et al. (2010) Scand. J. Immunol. 84: 215-251).

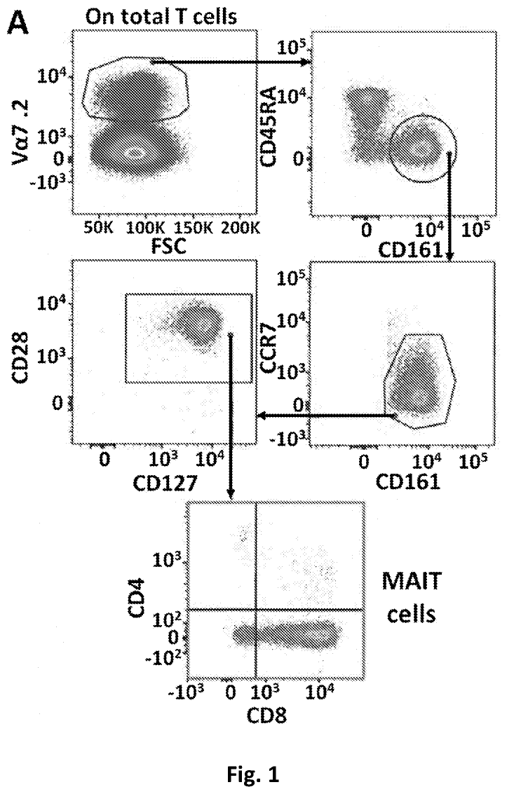

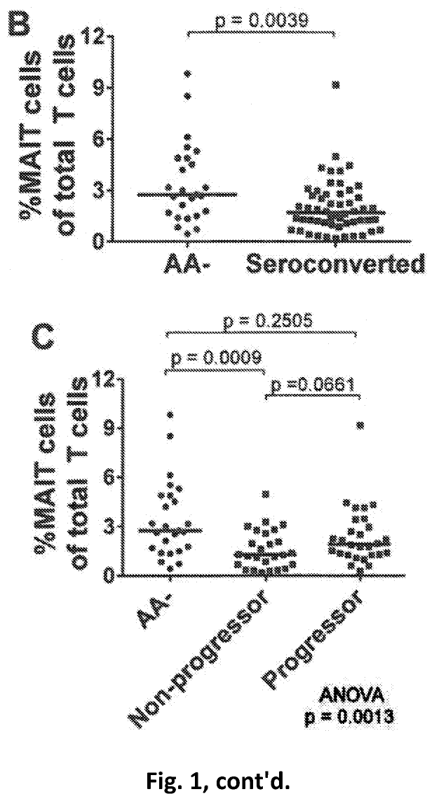

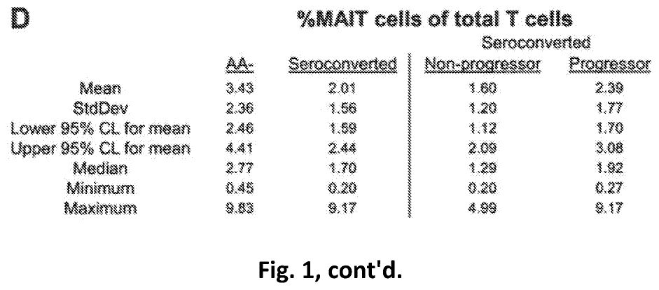

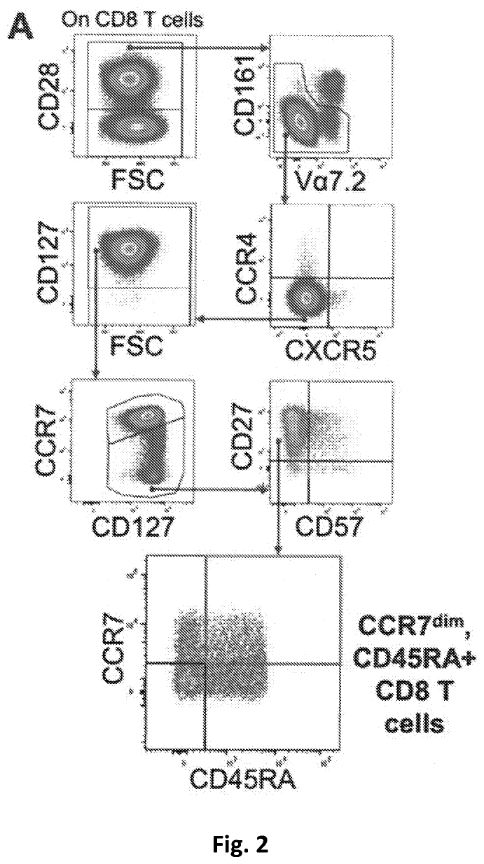

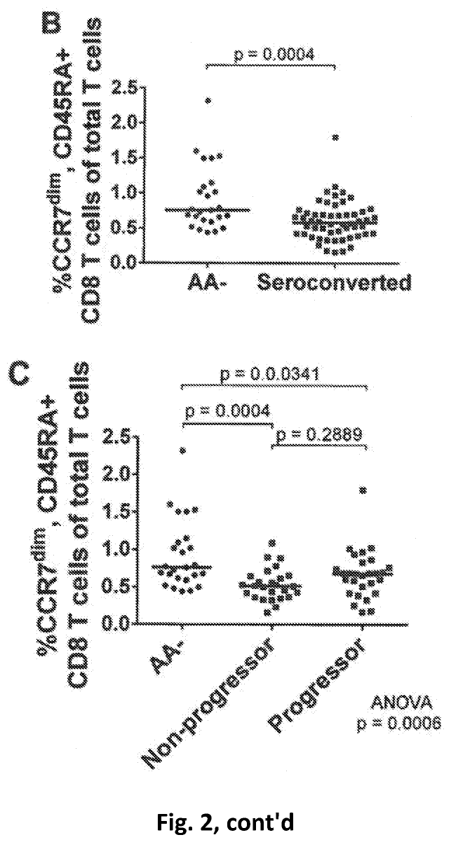

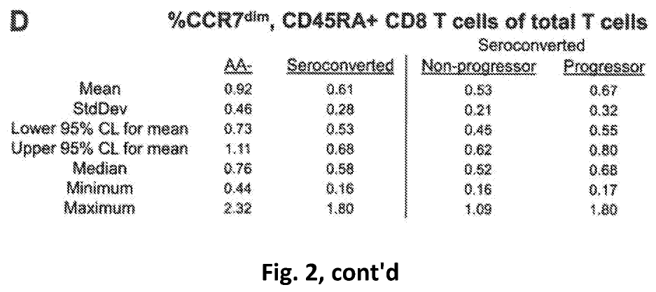

We identified total MAIT cells as CD3+, Vα7.2+, CD45RA^{low}, CCR7^{low}, CD28+, CD127+ events and also divided them by CD4 and CD8 expression (FIG. 1, panel A). With this approach, we observed a sharply reduced frequency of MAIT cells among seroconverted individuals (FIG. 1, panels B and D). This reduction extended into all MAIT cell subsets as defined by CD4 and CD8 expression, and appeared most acute within the major compartments identified as CD8+ and DN (Supplemental FIG. 2, panels A and B). When evaluating the seroconverted subjects according to disease progression, we found the non-progressors harbored the fewest MAIT cells, while the reduction among the progressors was not significant (FIG. 1, panels C and D). These data indicate that a MAIT cell reduction is associated with seroconversion and this effect is slightly ameliorated among those who progress to disease.

Short-Lived Effector-Like CD8 T Cells are Expanded in Seroconverted Individuals and this is Most Pronounced Among Those Who Progressed to Disease.

Conventional αβ CD8 T cells are the chief cytotoxic arm of adaptive immunity. Upon licensing and activation. CD8 T cells rapidly proliferate in order to destroy microbially-infected or transformed cells (Zhang & Bevan (2011) Immunity, 35: 161-168). Following the eradication of targets, effector cells die off while memory populations remain in tissue and circulation. This memory provides a quick recall response to repeat offenders and is considered a hallmark of T cell immunity. Outside of this role as defender, CD8 T cells have been incriminated in the pathogenesis of several autoimmune diseases (29).

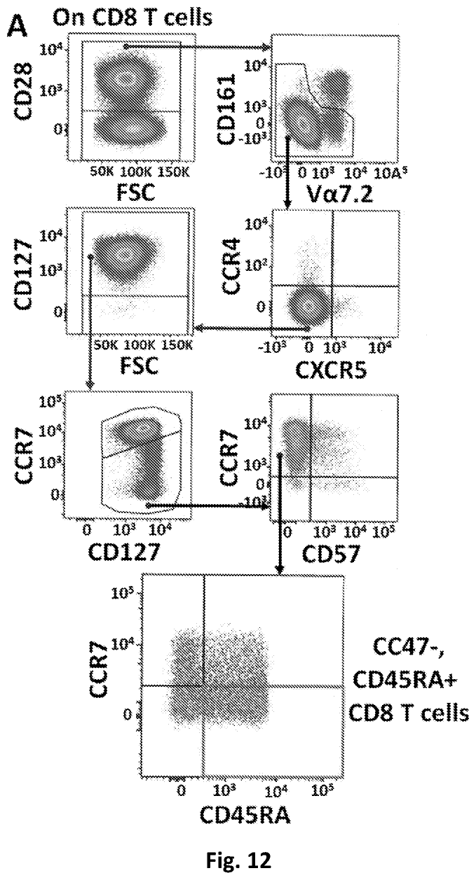

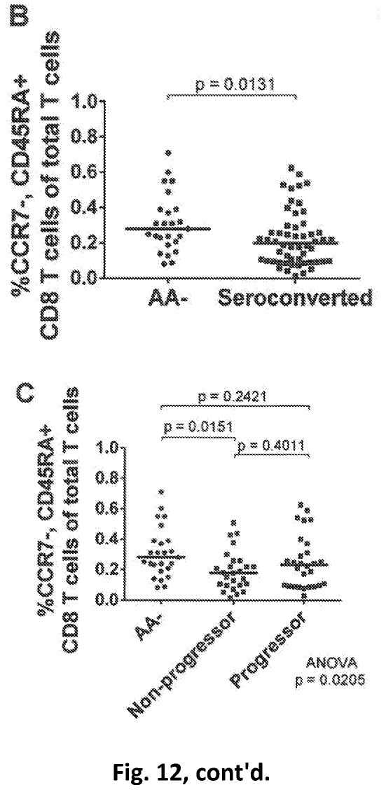

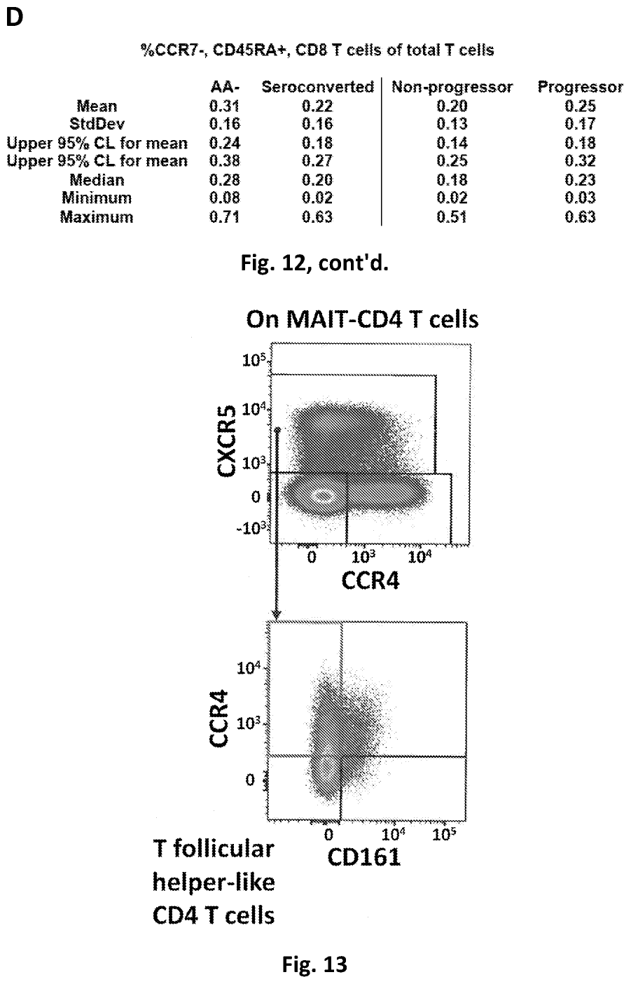

In our analysis of CD8 T cells, we first divided CD8 T cells according to presence or absence of costimulatory protein CD28. Among the CD28+CD8 T cells, we removed MAIT cells and analyzed the remaining CD8 T cells for expression levels of CCR4, CXCR5, CD45RA, CD27, CD127, CCR7, and CD57. Similar to what was observed among MAIT cells, seroconverted subjects possessed reduced proportions of CD45RA+, CCR7^{dim/-}, CD57−. CD27+, CD127^{high}, CXCR5−, CCR4− CD8 T cells compared to AA− subjects (FIG. 2, panels A, B, D, and FIG. 12). This combination of markers can best be described as a CD45RA+ memory subset following current differentiation paradigms (Inokuma et al. (2013) J. Immunol. Meth. 397: 8-17; Mahnke et al. (2013) Eur. J. Immunol., 43: 2797-2809). Upon dividing the seroconverted into non-progressor and progressor subsets, we found that the non-progressors were significantly reduced in proportion, while the progressors did not approach significance (FIG. 2, panels C and D and FIG. 12).

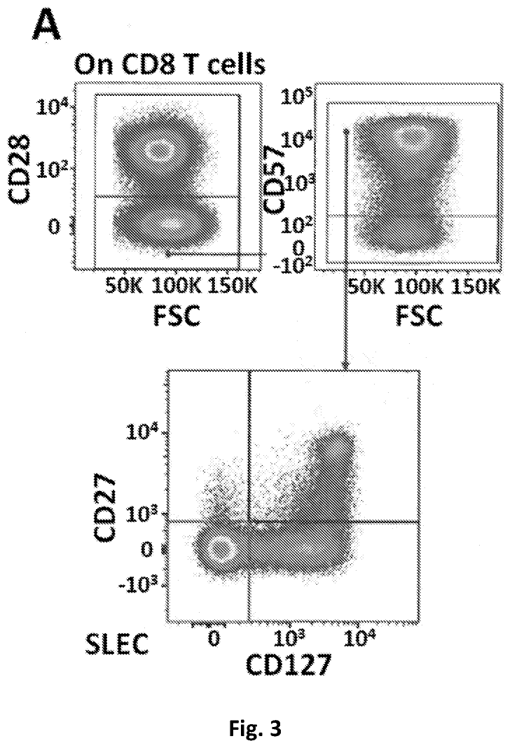

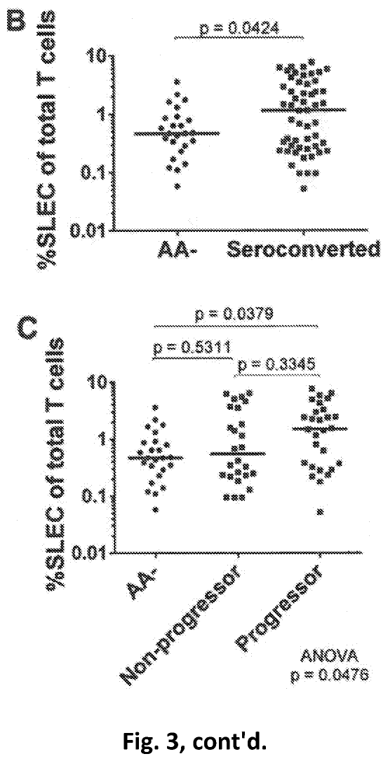

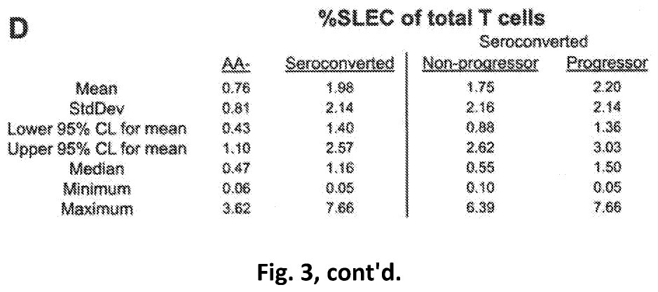

The absence of CD28 on human T cells is indicative of chronic stimulation (Vallejo (2005) Immunol. Rev. 205: 158-169). We divided CD28− CD8 T cells by expression of CD57, CD127, and CD27 to characterize this antigen-experienced compartment. Our analysis revealed an elevated frequency of CD127−, CD27−, CD57+, CD28− CD8 T cells among seroconverted subjects (FIG. 3, panels A, B, and D). This combined phenotype indicates terminal differentiation, cytotoxic potential via perforin production, and short-lived status (Borthwiek et al. (2000) Internat. Immunol. 12: 1005-1013; Cellerai et al. (2010) J. Virol. 84: 3868-3878; Chattopadhyay et al. (2009) J. Leukocyte Biol. 85: 88-97; Tomiyama et al. (2002) J Immunol. 168: 5538-5550). Thus, we abbreviated their phenotype as SLEC for short-lived effector-like cells. Intriguingly, this expansion of SLEC was most prominent among progressors (FIG. 3, panels C and D), suggesting an acute pathogen response may be associated with disease progression.

CCR4-Expressing CD4 T Cell Subsets, Including CD127^{dim} Treg-Like Cells, are Reduced in Seroconverted Subjects.

C-C chemokine receptor 4 (CCR4) expression among CD4 T cells suggests previous T cell receptor engagement (Singh et al. (2010) Eur. J. Immunol. 40: 3183-3197; Song et al. (2005) Proc. Natl. Acad. Sci. USA, 102: 7916-7921) as well as chemotactic responsiveness to thymus and activation-regulated chemokine (TARC), macrophage-derived chemokine (MDC), and chemokine like factor I (CKLF1) (Imai et al. (1997) J Biol. Chem. 272: 15036-15042; Imai et al. (1998) J. Biol. Chem. 273: 1764-1768; Wang et al. (2006) Life Sci. 78: 614-621). Rather than solely indicating “Th2” status, CCR4-expressing CD4 T cells can be enriched for IFN-γ, IL-22, IL-17 and/or IL-4 production, as well as possess regulatory function (Acosta-Rodriguez et al. (2007) Nat. Immunol. 8: 639; Baatar et al. (2007) J. Immunol. 178: 4891-4900; Duhen et al. (2009) Nat. Immunol. 10: 857; Kim et al. (2001) J. Clin. Invest. 108: 1331; Nanki & Lipsky (2000) Internat. Immunol. 12: 1659-1667).

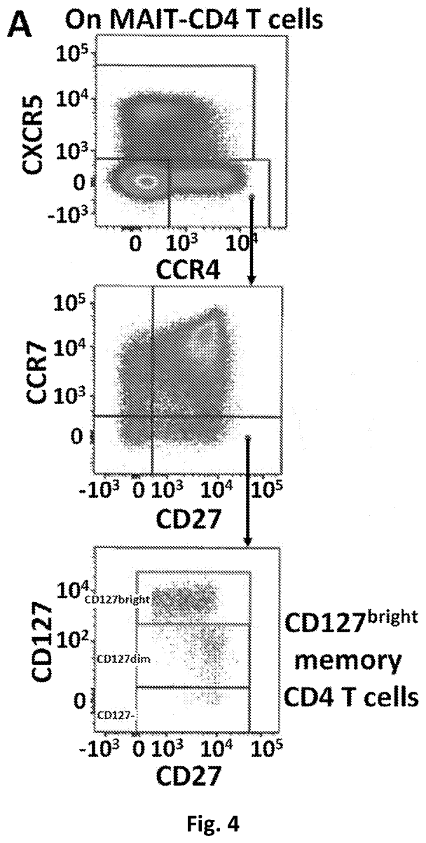

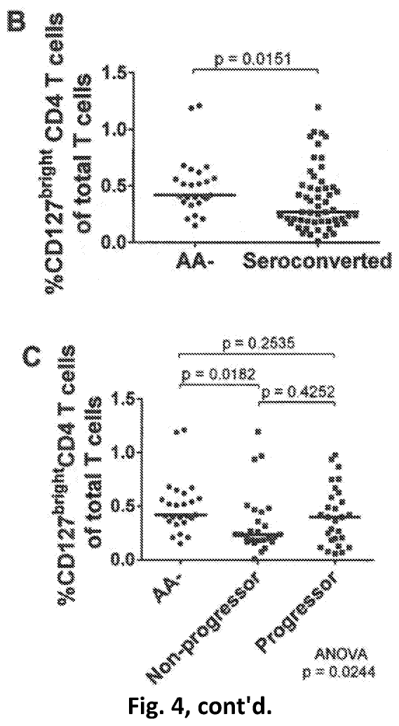

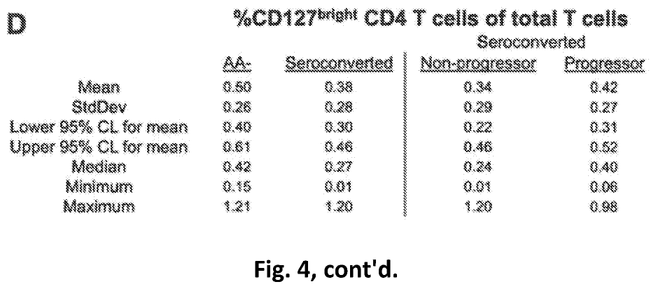

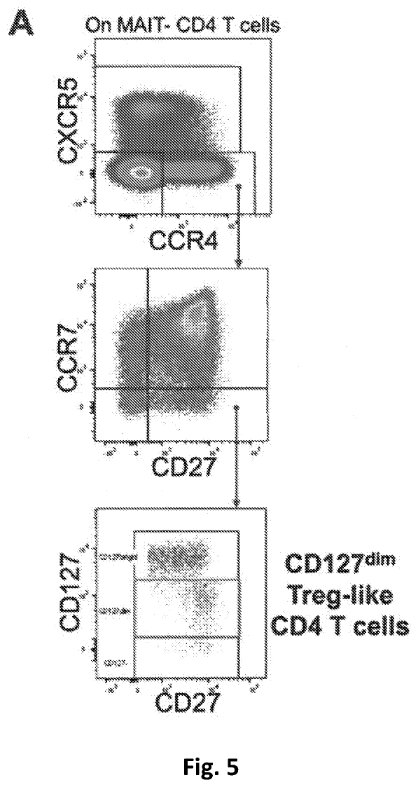

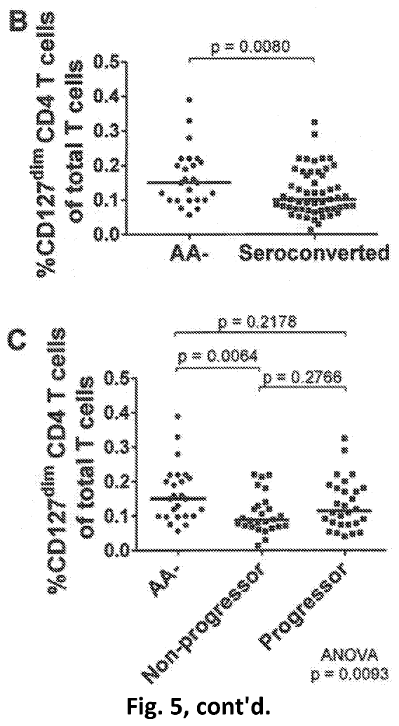

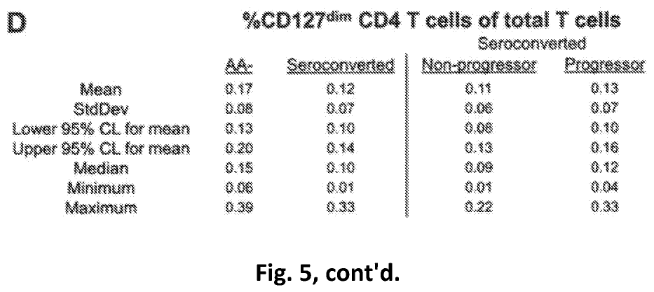

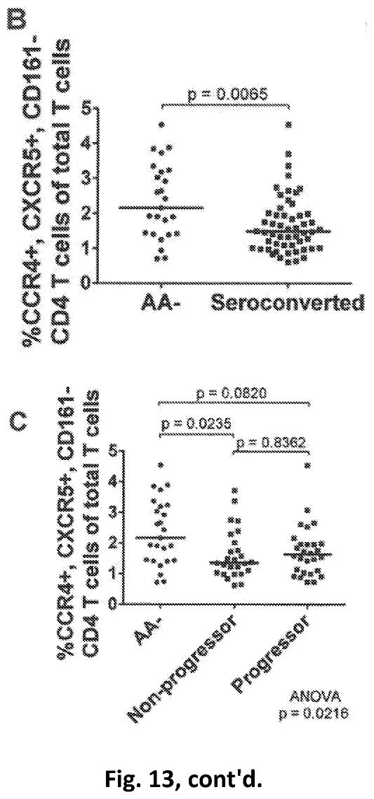

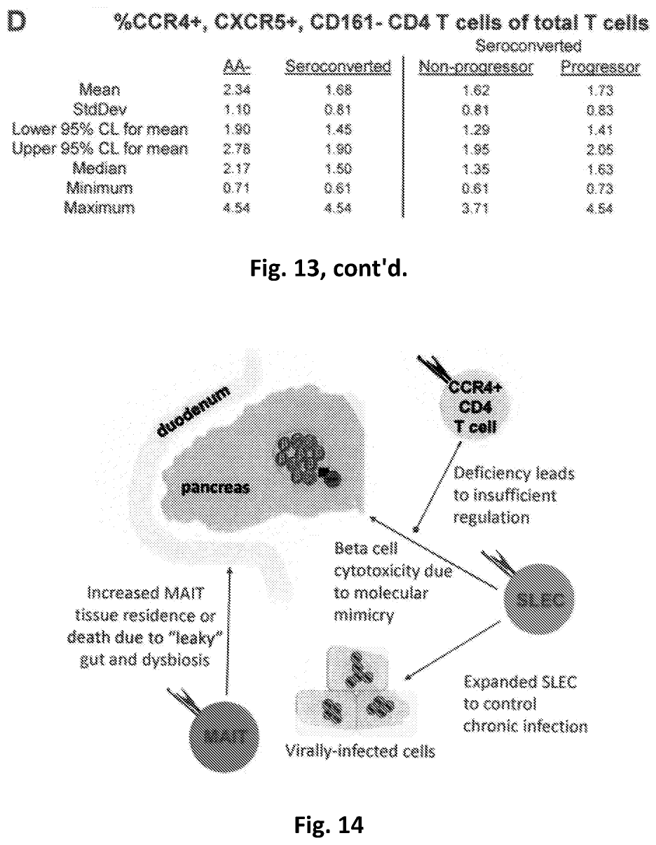

Our analysis of CD4 T cells from TrialNet donors revealed reductions in frequency of five CCR4-expressing subsets among seroconverted subjects. Memory (CD127^{bright}, CD27+, CCR7−, CCR4+, CXCR5− (FIG. 4, panels A-D). Treg-like (CD127^{dim}, CD27+, CCR7−, CCR4+, CXCR5− (FIG. 5, panels A-D), and T follicular helper-like (CCR4+, CXCR5+, CD161−; FIG. 13) CD4 T cell subsets were all reduced among seroconverted subjects. For each compartment, the reduction in frequency was most profound among non-progressors and approached “normal” levels among progressors.

As our T cell panel did not include CD25 nor FoxP3, we were unable to cross-confirm regulatory phenotype among the Treg-like, CCR4-expressing CD4 T cells that we observed contracted among TrialNet samples. However, we analyzed CD4 T cells from 3 healthy human donors and found that the CCR4+, CXCR5− compartment possesses a substantial FoxP3+, CD25+ subset, which is also CD127^{dim/-}, a combined phenotype indicating regulatory potential (FIG. 6). This is in agreement with previous studies indicating CCR4+ and CD127^{dim/low} CD4 T cells contain Tregs (Klein et al. (2010) Invest. Dermatol. 130: 492-499; Liu et al. (2006) J. Exp. Med. 203: 1701-1711; Sugiyama et al. (2013) Proc. Natl. Acad. Sci. USA, 110: 17945-17950). Thus, it's highly likely that the observed Treg-like CCR4+CD4 T cells we found to be reduced among seroconverted subjects contains T cells with regulatory potential.

Frequencies of CD127^{dim} Treg-Like Cells are Positively Correlated with CD127^{bright} Memory CD4 T Cells and with CD45RA+, CCR7^{dim} Memory CD8 T Cells Among Seroconverted Subjects.

We observed altered frequencies of MAIT, CCR7^{dim/-}, CD45RA+, CD8 memory, SLEC, and CCR4+CD4 T cells, including memory and Treg-like subsets. As mentioned previously, it's widely presumed that T1D is driven by autoreactive CD8 T cells which have escaped tolerance. Since Tregs can effectively suppress T cell proliferation in vivo (Putnam et al. (2009) Diabetes, 58: 652-662), it's plausible that such subsets may impact T cell expansions and contractions in vivo. Therefore, we examined some of the CD4 and CD8 T cell subsets described above for relationships with our CD127^{dim} Treg-like subset.

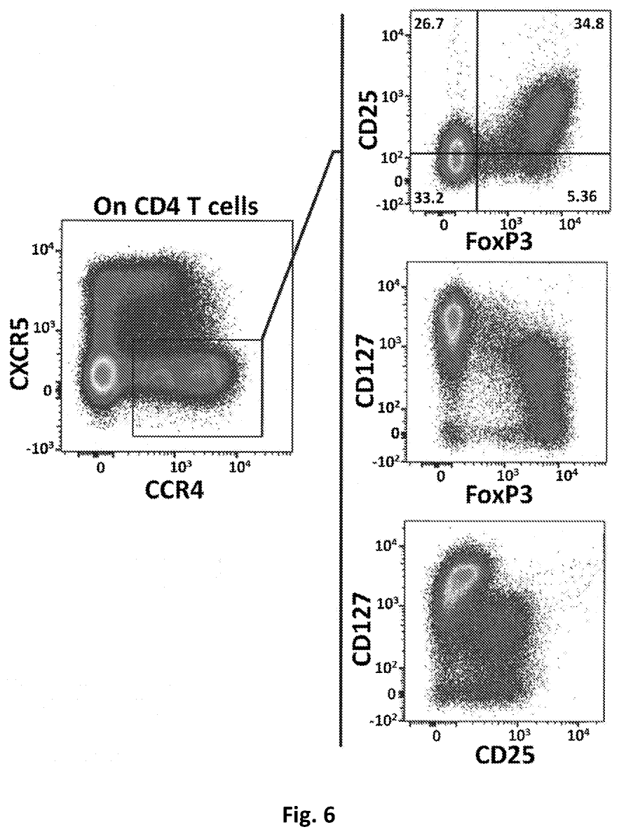

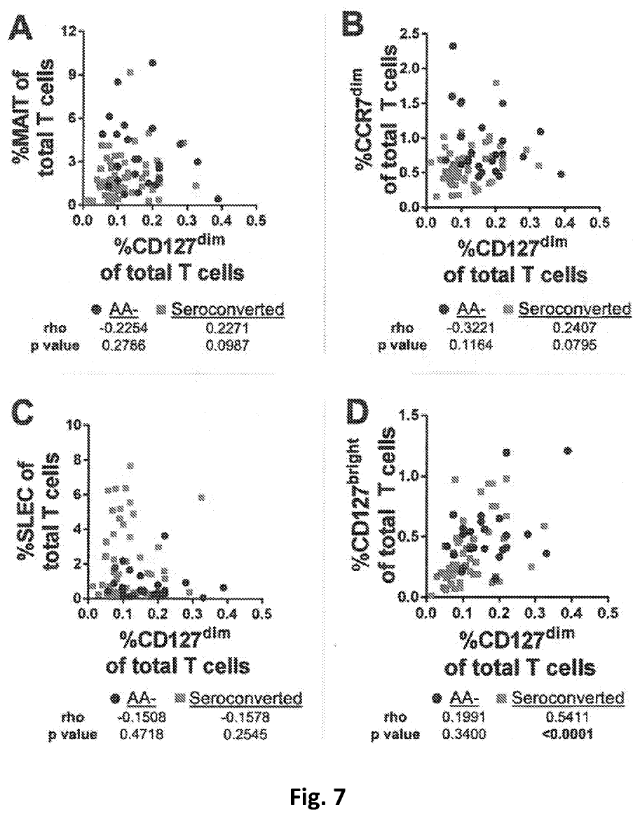

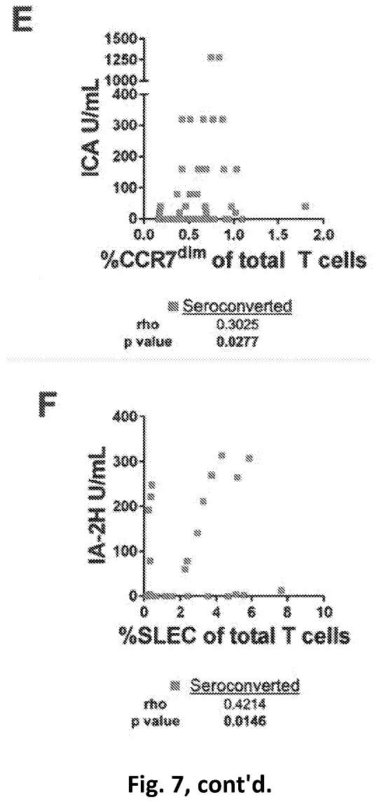

First, we compared frequencies of T cell subsets using Spearman's rank order correlation. We observed no significant correlations among CD127^{dim} Treg-like CD4 T cell cells with either MAIT cells or with SLEC CD8 T cells (FIG. 7, panels A and D). Alternatively. CD127^{bright} memory CD4 T cells were positively and significantly correlated with the frequency of CD127^{dim} Treg-like CD4 T cells, while there was no significant relationship among AA− subjects (FIG. 7, panel B). Further division of the seroconverted group into non-progressor and progressor demonstrated similar strong and significant positive correlations among both groups (data not shown). In total, these correlation data suggest that the frequency dynamics of the CD127^{dim} Treg-like cells do not negatively regulate frequencies of the noted T cell compartments in the periphery. However, they do demonstrate comparable and unique dynamics for CD127^{bright} memory CD4 T cells and CD127^{dim} CD4 T cells among only the seroconverted subjects. This could be due to similar effects driving the reductions observed in these three subsets.

In addition to the correlations comparing frequency of T cell subsets, we also examined relationships between autoantibody levels and frequency of T cells. Such a correlation could reveal if any of these subsets are associated with islet immunity. Interestingly, we observed a positive and significant correlation with islet cell antibody (ICA) values and the CCR7^{dim} CD45RA+, CD8 T cell subset (FIG. 7, panel E). Furthermore we found that the SLEC subset was positively and significantly correlated with harmonized insulinoma antigen 2 (IA-2H) levels (FIG. 7, panel F). We observed no significant correlations among other T cell subset with autoantibody levels (data not shown).

Seroconverted Subjects have Significantly Elevated Ratios of SLEC to Both CD127^{dim} Treg-Like Cells and MAIT Cells.

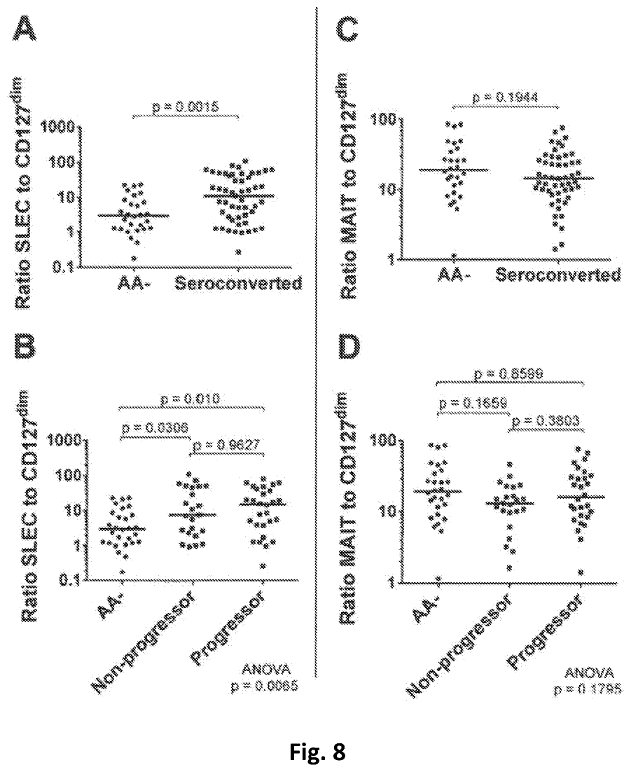

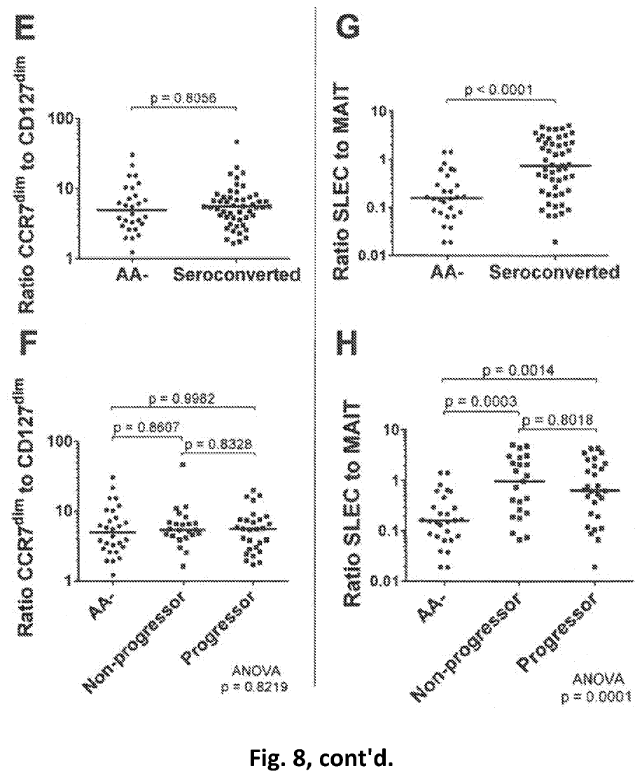

Along with these correlations, we examined ratios of frequency of effectors to frequency of regulators. Here, MAIT cells were hypothesized to function as either effector or regulator, as a recent report has suggested MAIT cells may have a regulatory function (Rouxel et al. (2017) Nat. Immunol. 18: 1321). We observed that the ratios of SLEC to CD127^{dim} or MAIT cells was significantly elevated among seroconverted, as well as when divided among non-progressors and progressors (FIG. 8, panels A, B, G, and H). Alternatively, the ratio of MAIT cells to CD127^{dim} was relatively similar when comparing AA− and seroconverted subjects, including progressors and non-progressors (FIG. 8, panels C and D). The ratio of CCR7^{dim}, CD45RA+CD8 T cells to CD127^{dim} CD4 T cells was also similar among AA− and seroconverted subjects, and this continued when seroconverted subjects were divided into non-progressors and progressors (FIG. 8, panels E and F).

SLEC Frequencies are Strongly Associated with CMV IgG Levels Among Seroconverted Subjects and CMV+ Progressors Harbor the Highest Frequencies of SLEC and Associated Chronically-Activated CD8 T Cells.

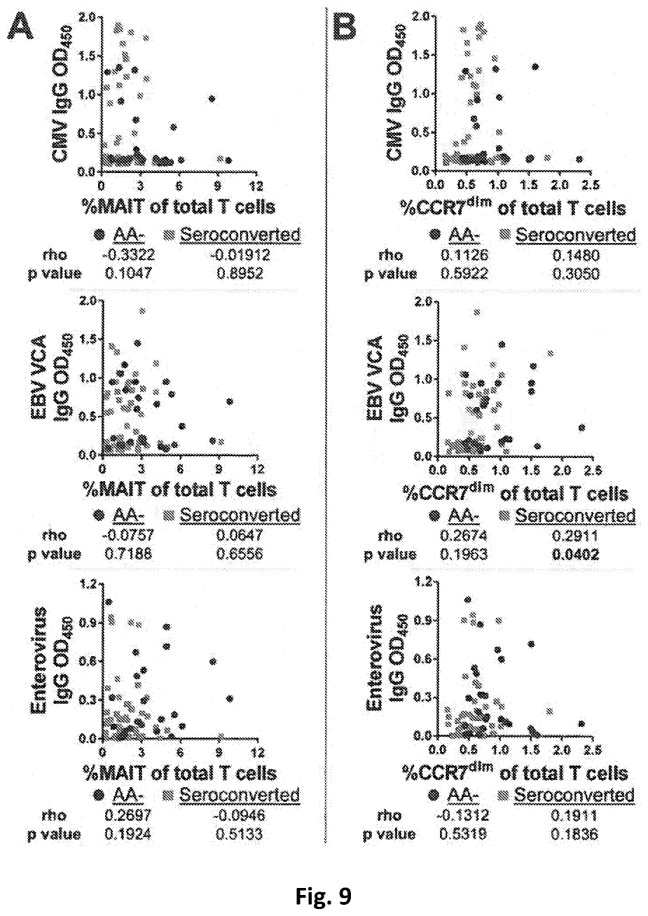

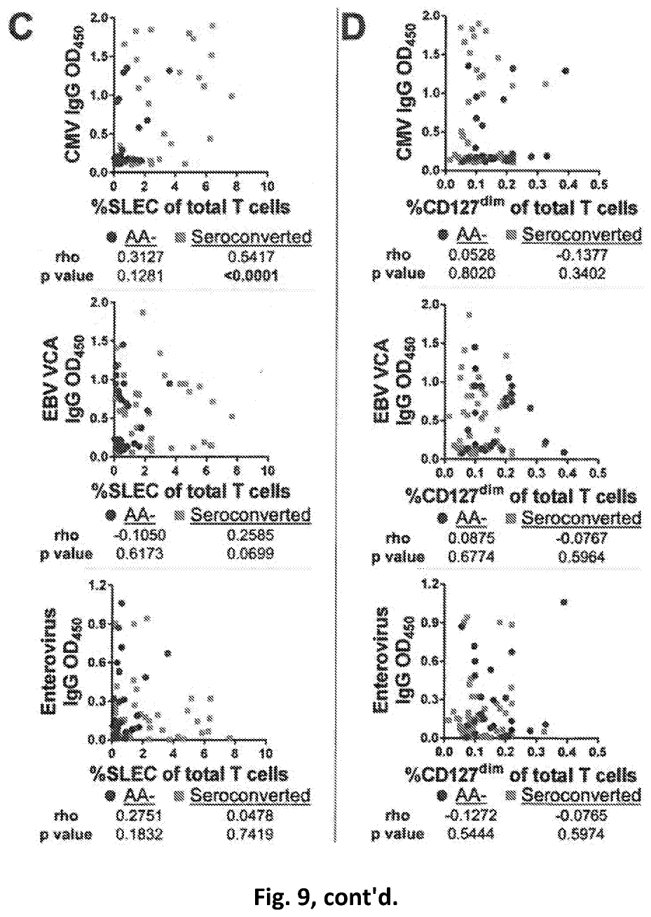

To determine if viral exposure was influencing the T cell changes associated with seroconversion, we tested plasma for abundance of anti-viral immunoglobulins using ELISA. We then performed correlations comparing T cell frequency with relative abundance of viral immunoglobulins as gauged by optical density values. Neither MAIT cells nor CD127^{dim} Treg-like cells were correlated with abundance of CMV, EBV VCA and enterovirus IgG (FIG. 9, panels A and D) nor IgM (data not shown). This was not the case for conventional CD8 T cells. We observed a weak but significant association between EBV VCA IgG and the CCR7^{dim} CD45RA+CD81 cells (FIG. 9, panel B). However, there was no significant association among this subset with CMV IgG or enterovirus IgG (FIG. 9, panel B), nor any viral IgM tested (data not shown). Finally, we observed a strong and significant association among SLEC and CMV IgG, but not with EBV VCA IgG, enterovirus IgG, nor any viral IgM tested (FIG. 9, panel C and data not shown).

Since the SLEC subset appeared to be strongly influenced by CMV, we stratified our subjects according to CMV IgG positivity and then retested the entirety of the CD28− CD8 T cell compartment by ANOVA. We observed that the SLEC expansion is greatest among CMV+ progressors (FIG. 10, panel A). Furthermore, we observed that the CMV+ progressors possessed elevated frequencies of additional chronically-stimulated CD28− subsets (FIG. 10, panels B-E). While these lacked the expression of CD57, they were heterogeneous for CD127 and CD27 expression. In total, these data reveal a relationship among type 1 diabetes progression, terminally-differentiated effector-like CD8 T cells and existing cytomegalovirus infection among at-risk, seroconverted subjects.

### Discussion

The etiology of type 1 diabetes remains obscure. This is due, in part, to the inaccessibility of the target tissue. Indeed, pancreatic biopsy currently comes with unwanted risk for the donor (Krogvold et al. (2014) Diabetologia, 57: 841-843). Analysis of circulating white blood cells as a proxy for solid tissue interrogation allows us to apparently circumvent the problem of pancreas accessibility. Thus, our cross-sectional study was designed to screen multiple T cell subsets from at-risk subjects in order to reveal if immune perturbations are associated with seroconversion and disease progression. To that end, the answer is a clear “yes”. However, this “indirect” analysis is plagued by at least two deficiencies. First, in the absence of a defined causal factor (or set of factors), increases/decreases in frequency of circulating immune cells cannot be explained satisfactorily (Blunt & Pabst (2007) Imunol. Letts. 108: 45-51; Westermann & Pabst (1990) Immunol. Today, 11: 406-410). Second, without follow-up longitudinal analysis of these subjects, such changes in frequency cannot be understood in their dynamic processional roles through time from seroconversion to diagnosis. With these concerns in mind, the following discussion will focus on relevant available reports on comparable immune cell subsets as mere possibilities, as fundamental mechanistic explanations are not available.

Due to their unique function, MAIT cells have been examined in several disease states. Among T1D, the findings are not consistent. We have previously found no change in proportion or number of CD8+ MAIT cells front diabetics in relation to controls (Harms et al. (2015) PLos One, 10: e0117335). Furthermore, we have recently replicated and extended these findings among a second cohort of age-matched juvenile TlDs and controls. Alternatively. Rouxel and colleagues reported reduced frequencies of total MAIT cells to be associated with T1D (Rouxel et al. (2017) Nat. Immunol. 18: 1321). In the same study, they observed no reduction in proportion of total MAIT cells in their examination of small cohort of at-risk subjects acquired from TrialNet. Whether these discrepancies result from methodological and/or sampling differences is unclear and independent studies must he conducted for clarification.

Aside front T1D, reductions in circulating MAIT cell populations have been associated with enteric fever (Howson et al. (2018) Nat. Comm. 9: 253), several viral infections (Cosgrove et al. (2013) Blood, 121: 951-961; Loh et al. (2016) Proc. Natl. Acad. Sci. USA, 113: 10133-10138; Paquin-Proulx et al. (2017) PLoS One, 12: e0175345; Van Wilgenburg et al. (2016) Nat. Comm. 7: 11653), and among other autoimmune diseases and inflammatory disorders such as MS, SLE, RA, and IBD (Cho et al. (2014) J Immunol. 193: 3891-3901; Hiejima et al. (2015) Inflam. Bowel Dis. 21: 1529-1540; Kim et al. (2017) Cytokine, 99: 91-98; Willing et al. (2014) Eur. J Immunol. 44: 3119-3128). Such studies have indicated that MAIT cells are prone to apoptosis in inflammatory milieus, and that reductions in the periphery may co-occur with elevations or reductions in other tissues (Cosgrove et al. (2013) Blood, 121: 951-961; Hiejima et al. (2015) Inflam. Bowel Dis. 21: 1529-1540; Kim et al. (2017) Cytokine, 99: 91-98; Willing et al. (2014) Eur. J. Immunol. 44: 3119-3128; Dunne et al. (2013) PloS One, 8: e76008). Importantly, type I interferons, perturbed microbiota, viral infection, and increased intestinal permeability have all been associated with the development of T1D (Bosi et al. (2006) Diabetologia, 49: 2824-2827; Ferreira et al. (2014) Diabetes, 63(7): 2538-2550; Kostic et al. (2015) Cell Host & Microb. 17: 260-273; Krogvold et al. (2014) Diabetes, 64(5): DOI: 10.2337/db14-1370). It remains to be determined if and how these factors impact the MAIT cell lineage and if they are contributing to the deficiencies we have observed.

Along with reduced frequencies of MAIT cells, we observed reductions in several CD4 T cell subsets, which, although phenotypically diverse, all share the expression of CCR4. This is not the first report of CCR4 alterations in T1D. A reduced frequency of CCR4+CD3+ cells was observed among recently-diagnosed type 1 diabetics (Lohmann et al. (2002) Diabetes, 51: 2474-2480). Additionally. reduced CCR4 expression following Th1 or Th2 polarization was observed among cord blood T cells from individuals possessing genetic risk alleles for T1D (Luopajarvi et al. (2007) Immunol. 121: 189-196). These intriguing results suggest that the CCR4 deficiencies we have observed among seroconverted subjects may have a genetic component and ultimately originate during thymic selection.

At this point, we cannot define functional roles for the CCR4− expressing subsets we found to be diminished. As described above, there is justification to think that regulatory cells are residing in the CD127^{dim} compartment. Nevertheless, a recent examination of TrialNet samples enquiring into FOXP3 and IL-17-producing CD4 T cells did not report reduced frequencies (Marwaha et al. (2017) Genes & Immun. 18: 15). Due to key methodological differences, it's uncertain how much to extend from this study onto ours. Yet, should we presume that seroconverted subjects are not deficient in regulatory or Th17 cells (both of which are plausible based upon phenotype), we are left with likely Th1 or Th22-type cells. Although relatively unexplored in T1D, Th22-type CD4 T cells have been found in the human gut mucosa (Femandes et al. (2014) J Infect. Dis. 210: 630-640; Kim et al. (2012) Mucosa/Immunol. 5: 670) and the cytokine IL-22 plays a key role in intestinal defense and integrity (Sonnenberg et al. (2010.) Adv. Immunol. 107: 1-29). Alternatively, the Th2 lineage is generally associated with anti-parasitic and allergic responses (Harris et al. (2017) Immunity, 47: 1024-1036). Interestingly, part of the hygiene hypothesis suggests that the absence of a manageable parasite load is a factor driving increases in the prevalence of autoimmunity (Rook (2012) Clin. Rev. Allergy & Immunol. 42: 5-15).

While subset functional status will remain elusive for the time being, the expression of CCR4 clearly indicates responsiveness to MDC, TARC, and CKLF1. Accordingly plasma from TrialNet subjects can be evaluated for abundance of these and other factors. Importantly, there is some evidence that MDC may play a role in T1D. Elevated MDC transcripts have been found in the duodenal mucosa among T1Ds (Pellegrini et al. (2017) J Clin. Endocrin. Metab. 102: 1468-1477) and MDC can be produced by human islets exposed to a cocktail of inflammatory cytokines (Sarkar et al. (2012) Diabetes, 61: 436-446).

Among conventional CD8 T cells, our screening for T cell aberrations revealed two distinct compartments both associated with islet autoantibody levels. At this point, little can be said regarding the reduced frequency of the CD45RA+, CCR7^{dim/-} CD8 T cell subsets. While their precise characterization indicates central effector memory-type CD8 T cells, discussions of ontogeny, functionality or specificity of these subsets would be purely speculative and tantamount to hand-waving. Nevertheless, some compelling observations associated with this group do stand out for comment. First, the correlation with ICA levels associates these T cells with beta cell destruction yet the populations are not expanded among seroconverted subjects and are most reduced among non-progressors. This implies that the “normal” frequencies observed among the progressors could be cryptically pathogenic. Second, the modest association with EBV VCA IgG levels is notable, especially in light of a recent report of a causal role for EBV in autoimmunity (Harley et al. 2018) Nat. Genet. 50: 699). Stratification of our subjects according to EBV positivity, as we performed for CMV, did not reveal significant associations with disease progression (data not shown), therefore the overall impact of EBV on the development of disease at this point is uncertain.

Fortunately, we can be somewhat more definite in the discussion of the SLEC population, whose phenotype (CD28−, CD57+, CD27−, and CD127−) is indicative of effector status and cytotoxic potential. Furthermore, our data demonstrate that the SLEC subset is strongly associated with CMV IgG levels. Comparable phenotypes have been associated with CMV infection previously (Appay et al. (2002) Nat. Med. 8: 379; Boutboul et al. (2005) Aids, 19: 1981-1986; Hoji et al. (2007) Clin. & Vaccine Immunol. 14: 74-80; Jensen et al. (2016) JAIDS J Acquired Immune Def Synd. 71: 8-16; Paiardini et al. (2005) J Immunol. 174: 2900-2909; Raciszadeh et al. (2006) Clin & Exp. Immunol. 146: 234-242; Weekes et al. (1999) Immunol. 98: 443-449). From our stratifications, we observed that CMV+ progressors possess much higher frequencies of SLEC and related subsets in comparison to CMV+AA− and non-progressor subjects. This novel observation links a cytotoxic CD8 T cell subset with disease progression and viral exposure. One remaining question is what is driving this cellular expansion. One possibility is an acute response to an active CMV infection. To that end, we did not observe CMV IgM positivity among any of the subjects in this study. Nevertheless, we cannot rule out an active viral infection since we were unable to analyze plasma or circulating leukocytes for presence and abundance of viral nucleic acids (Ross et al. (2011) Infect. Disorders Drug Targets (Formerly Current Drug Targets-Finectious Disorders), 11: 466-474). Such an analysis remains a priority.

In the absence of an active CMV infection, alternate possibilities exist. Following the initial infection. CMV establishes latency and a lifelong residence in the host (Sinclair (2008) J Clin. Virol. 41: 180-185). Over time, reactivation from latency is presumed to drive the expansion of responding CD8 T cells due to chronic antigen exposure (Komatsu et al. (2006) Immunity & Aging, 3: 11; Lee (2014) PLoS One, 9: e89444). In this scenario, the SLEC expansion may represent an exhausted T cell response due to elevated or uncontrolled reactivation of CMV over time. How this could impact beta cells is unknown. Importantly, we observed a positive correlation with IA-2 levels and SLEC frequencies among seroconverted subjects, thereby associating SLEC with beta cell demise and autoimmunity. Whether SLEC could be directly or indirectly responsible for beta cell death remains to be determined. There is in vitro evidence that CMV can infect beta cells (Smelt et al. (2012) Pancreas, 41: 39-49). However, pancreatitis is not normally associated with CMV infection (Wreghitt et al. (2003) Clin. Infect. Dis. 37: 1603-1606), making the direct lysis of CMV-infected beta cells less likely. Aside from direct infection, an answer could be found in the concept of molecular mimicry. It has been reported the CMV major capsid protein shares 64% identity and 73% similarity to the dominant epitope peptide of IA-2 (Honeyman et al. (1998) Mol. Med. 4: 231). Through that lens, one could imagine the inflated population we have observed to be a polyclonal pool of effectors, some “autoreactive”. Such an expansion could explain the precipitous decline in beta cells that has been observed in the progression to diagnosed diabetes (Herold et al. (2015) J. Clin. Invest. 125: 1163-1173), as well as the negative association among CMV-specific CD9 T cells and c-peptide levels among diagnosed type I diabetics (Yea et al. (2018) J. Clin. Invest. 128: 3460-3474).

In summary, the combined results of our T cell analysis among seroconverted subjects demonstrate reductions of innate-like, memory and regulatory subsets, with a clear expansion of an SLEC compartment (see, e.g., FIG. 14 for an interpretation of some of these phenomena). Intriguingly, while the expansion of the SLEC population is most prominent among the progressors, the remaining subsets appear to approach normal mean levels among those same subjects, albeit somewhat bimodally distributed. The causal foundations of these observations are not available. However, they do suggest that measurable and unique immunological changes occur as one moves toward clinical disease. Thus, assuming that seroconversion indicates a triggering event that led to beta cell destruction, all seroconverted subjects share a common history. Concomitantly, seroconverted subjects also have reduced frequencies of MAIT cells, CCR4+CD4 T cells, and CD45RA+ memory CD8 T cells. For progressors, the advance of beta cell destruction, which is generally foretold by increasing number of autoantibody specificities, is then also foretold by recurrence to relatively normal abundance of these T cell compartments. If, when, and how these populations contribute to autoimmunity remains to be determined. Nevertheless, weaving through this tapestry is the expanded, CMV-associated SLEC compartment, which is clearly present in progressors as well as some non-progressors. This expansion may then be viewed as a hit-and-run or remitting-and-relapsing response to pathogen and/or autoantigen, theories that have been proposed previously (Oldstone (1998) FASEB J. 12: 1255-1265; Von Herrath et al. (2007) Nat. Rev. Immunol. 7: 988).

While the paradigm described above could be considered “counterintuitive”, we are wary of talk of intuition in the absence of data. To that end, future efforts should focus on two complementary directions. One, longitudinal analysis of these subjects must be performed to delineate these population dynamics prior to seroconversion, at seroconversion, and closer toward diagnosis. Two, the characterization of the TCR specificity of the SLEC population, elucidation of the functional status of the CCR4-expressing CD4 T cells, and exploration of the fate of the MAIT cell lineage are essential to explicate how the immune system is responding during beta cell demise. In combination, such an approach should reveal which environmental determinants are driving T1D.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method of determining if a subject is at risk for progression to type 1 diabetes, said method comprising:
determining the level(s) of CD57+, CD28−, CD127−, CD27−, CD8+ T cells (SLEC CD8 T cells), and/or CD127−, CD27+, CD57−, CD28− CD8 T cells, and/or CD127−, CD27−, CD57−, CD28− CD8 T cells, and/or CD127+, CD27−, CD57−, CD28− CD8 T cells, and/or associated CD8 T cell subsets shown in FIG. 10, and/or CCR7^{dim}, CD45RA+, CD8+ T cells in blood or a blood fraction derived from said subject, where:
an elevated level of CD57+, CD28−, CD127−, CD27−, CD8+ T cells (SLEC CD8 T cells), and/or CD127−, CD27+, CD57−, CD28− CD8 T cells, and/or CD127−, CD27−, CD57−, CD28− CD8 T cells, and/or CD127+, CD27−, CD57−, CD28− CD8 T cells, and/or associated CD8 T cell subsets shown in FIG. 10 as compared to the level(s) of said T cells in a normal healthy control that does not progress to type 1 diabetes is an indicator that said subject has a significant risk for progression to type 1 diabetes; and/or
a reduced level CCR7^{dim}, CD45RA+, CD8+ T cells as compared to the level of said CCR7^{dim}, CD45RA+, CD8+ T cells T cells in a normal healthy control that does not progress to type 1 diabetes is an indicator that said subject has a significant risk for progression to type 1 diabetes.

2. The method of claim 1, wherein said method comprises determining the level of CD57+, CD28−, CD127−, CD27−, CD8+ T cells (SLEC CD8 T cells) in blood or a blood fraction derived from said subject, where an elevated level of SLEC CD8 T cells as compared to the level of SLEC CD8 T cells in a normal healthy control that does not progress to type 1 diabetes is an indicator that said subject has a significant risk for progression to type 1 diabetes.

3. The method according to any one of claims 1-2, wherein said method comprises determining the level of CD127−, CD27+, CD57−, CD28− CD8 T cells in blood or a blood fraction derived from said subject, where an elevated level of said CD127−, CD27+, CD57−, CD28− CD8 T cells T cells as compared to the level of said CD127−, CD27+, CD57−, CD28− CD8 T cells in a normal healthy control that does not progress to type 1 diabetes is an indicator that said subject has a significant risk for progression to type 1 diabetes.

4. The method according to any one of claims 1-3, wherein said method comprises determining the level of CD127−, CD27−, CD57−, CD28− CD8 T cells in blood or a blood fraction derived from said subject, where an elevated level of said CD127−, CD27−, CD57−, CD28− CD8 T cells T cells as compared to the level of said CD127−, CD27−, CD57−, CD28− CD8 T cells in a normal healthy control that does not progress to type 1 diabetes is an indicator that said subject has a significant risk for progression to type 1 diabetes.

5. The method according to any one of claims 1-4, wherein said method comprises determining the level of CD127+, CD27−, CD57−, CD28− CD8 T cells in blood or a blood fraction derived from said subject, where an elevated level of said CD127+, CD27−, CD57−, CD28− CD8 T cells T cells as compared to the level of said CD127+, CD27−, CD57−, CD28− CD8 T cells in a normal healthy control that does not progress to type 1 diabetes is an indicator that said subject has a significant risk for progression to type 1 diabetes.

6. The method of according to any one of claims 1-5, wherein said method comprises determining the level of CCR7^{dim}, CD45RA+, CD8+ T cells in blood or a blood fraction from said subject, where a reduced level of said CD8 T cells as compared to the level of said T cells in a normal healthy control that does not progress to type 1 diabetes is a further indicator that said subject has a significant risk for progression to type 1 diabetes.

7. The method according to any one of claims 1-6, wherein said elevated level and/or said reduced level is a statistically significant elevated level and/or a statistically significant reduced level compared to said normal healthy control.

8. The method of claim 7, wherein said elevated level and/or said reduced level is a statistically significant elevated level and/or a statistically significant reduced level compared to said normal healthy control(s) at a p value of p≤0.05, or p≤0.02, or p≤0.01.

9. The method according to any one of claims 1-8, wherein the level(s) of CD57+, CD28−, CD127−, CD27−, CD8+ T cells (SLEC CD8 T cells), and/or CD127−, CD27+, CD57−, CD28− CD8 T cells, and/or CD127−, CD27−, CD57−, CD28− CD8 T cells, and/or CD127+, CD27−, CD57−, CD28− CD8 T cells, and/or associated CD8 T cell subsets shown in FIG. 10, and/or CCR7^{dim}, CD45RA+, CD8+ T cells are determined by antibody labeling of T cells and flow cytometry.

10. The method according to any one of claims 1-9, wherein said subject is a subject that has been identified as seroconverted for autoantibodies.

11. The method of claim 10, wherein said subject is identified as seroconverted by screening blood or a blood fraction from said subject for antibodies directed against one of more biomarkers selected from the group consisting of GAD65, IA-2, and IAA.

12. The method of claim 11, wherein after a positive identification of antibodies against one or more of said biomarkers, blood or a blood fraction derived from said subject is screened for the biomarkers ZnT8 and/or ICA.

13. The method according to any one of claims 11-12, wherein positive thresholds for said biomarkers are: IAA>0.010, GAD65>0.032, ICA512>0.049, GAD65H>20; IA-2H>5, ZnT8>0.020 and/or ICA≥10.

14. The method according to any one of claims 1-13, wherein said subject has not progressed to clinical type 1 diabetes.

15. The method of claim 14, wherein said subject shows a normal hemoglobin A1c (HbA1c) level ranging from 4% to 5.6%.

16. The method of claim 14, wherein said subject shows an elevated hemoglobin A1c (HbA1c) level ranging from 5.7% to 6.4%.

17. The method of claim 14, wherein a random blood sugar test for said subject is below 200 mg/dL (11.1 mmol/L).

18. The method of claim 14, wherein a normal fasting blood sugar level for said subject is less than 100 mg/dL (5.6 mmol/L).

19. The method of claim 14, wherein an elevated fasting blood sugar level for said subject ranges from 100 to 125 mg/dL (5.6 to 6.9 mmol/L).

20. The method according to any one of claims 1-19, wherein said subject is evaluated for the presence of an active viral infection.

21. The method of claim 20, where said subject is evaluated for an active viral infection by screening for viral IgM positivity, and/or analyzing plasma or circulating leukocytes for presence and abundance of viral nucleic acids.

22. The method according to any one of claims 20-21, wherein said viral infection is a CMV or other Herpesvirus infection.

23. The method according to any one of claims 1-22, wherein the level of CD57+, CD28−, CD127−, CD27−, CD8+ T cells (SLEC CD8 T cells), and/or CD127−, CD27+, CD57−, CD28− CD8 T cells, and/or CD127−, CD27−, CD57−, CD28− CD8 T cells, and/or CD127+, CD27−, CD57−, CD28− CD8 T cells, and/or associated CD8 T cell subsets shown in FIG. 10, and/or CCR7^{dim}, CD45RA+, CD8+ T cells and/or a diagnosis/prognosis based, at least in part, on said levels is recorded in a medical record for said subject.

24. The method according to any one of claims 1-23, wherein the level of level of CCR7^{dim}, CD45RA+, CD8+ T cells and/or a diagnosis/prognosis based, at least in part, on said levels is recorded in a medical record for said subject.

25. The method according to any one of claims 23-24, wherein said medical record is maintained by a laboratory, physician's office, a hospital, a health maintenance organization, an insurance company, or a personal medical record website.

26. The method according to any one of claims 1-25, wherein a diagnosis, based at least in part on the level(s) of CD57+, CD28−, CD127−, CD27−, CD8+ T cells (SLEC CD8 T cells), and/or CD127−, CD27+, CD57−, CD28− CD8 T cells, and/or CD127−, CD27−, CD57−, CD28− CD8 T cells, and/or CD127+, CD27−, CD57−, CD28− CD8 T cells, and/or associated CD8 T cell subsets shown in FIG. 10, and/or CCR7^{dim}, CD45RA+, CD8+ T cells is recorded on or in a medic alert article selected from a card, worn article, or radiofrequency identification (RFID) tag.

27. The method according to any one of claims 23-26, wherein the level(s) of CD57+, CD28−, CD127−, CD27−, CD8+ T cells (SLEC CD8 T cells), and/or CD127−, CD27+, CD57−, CD28− CD8 T cells, and/or CD127−, CD27−, CD57−, CD28− CD8 T cells, and/or CD127+, CD27−, CD57−, CD28− CD8 T cells, and/or associated CD8 T cell subsets shown in FIG. 10, and/or CCR7^{dim}, CD45RA+, CD8+ T cells and/or a diagnosis based upon said levels is recorded on a non-transient computer readable medium.

28. The method according to any one of claims 1-27, wherein the level(s) of CD57+, CD28−, CD127−, CD27−, CD8+ T cells (SLEC CD8 T cells), and/or CD127−, CD27+, CD57−, CD28− CD8 T cells, and/or CD127−, CD27−, CD57−, CD28− CD8 T cells, and/or CD127+, CD27−, CD57−, CD28− CD8 T cells, and/or associated CD8 T cell subsets shown in FIG. 10, and/or CCR7^{dim}, CD45RA+, CD8+ T cells are determined as part of a differential diagnosis.

29. The method according to any one of claims 1-28, wherein said subject is a human and said blood or blood fraction is from said human.

30. The method according to any one of claims 1-29, wherein when said subject is identified as at significant risk for progression to type 1 diabetes the subject is provided treatment to slow or prevent the onset of type 1 diabetes.

31. The method of claim 30, wherein said treatment comprises administering an immune modulator to said subject.

32. The method of claim 31, wherein said immune modulator comprises an anti-viral agent, an anti-PD1 antibody, an anti-CTLA4 antibody, and/or an anti-CD3 antibody.

33. The method of claim 32, wherein said immune modulator comprises an anti-CD3 monoclonal antibody.

34. The method of claim 33, wherein said immune modulator comprises teplizumab.

35. The method of claim 32, wherein said immune modulator comprises an anti-viral agent.

36. The method of claim 35, wherein said immune modulator comprises an anti-viral agent selected from the group consisting of Pleconaril and Ribavirin.

37. The method according to any one of claims 30-36, wherein said method comprises re-induction of tolerance towards the putative self-antigen that causes T1D.

38. The method of claim 37, wherein said antigen comprises one or more antigen selected from the group consisting of insulin, glutamic acid decarboxylase (GAD), and the heat shock protein 60 (Hsp60)-derived peptide 277.

39. The method according to any one of claims 30-38, wherein said treatment comprises monitoring blood glucose levels in said subject.

40. The method of claim 39, wherein said blood glucose is tested before meals and snacks, and/or before bed, and/or before exercising or driving.

41. The method of claim 39, wherein said blood glucose is monitored on a daily, a weekly, a biweekly, or a monthly basis.

42. The method according to any one of claims 30-41, wherein said treatment comprises fitting said subject with a continuous glucose monitoring system.

43. The method according to any one of claims 30-42, wherein said treatment comprises monitoring hemoglobin A1c.

44. The method according to any one of claims 30-43, wherein said treatment comprise maintaining normal blood glucose levels.

45. The method of claim 44, wherein said maintaining normal blood glucose levels comprises administering insulin.

46. The method of claim 45, wherein said insulin comprises an insulin selected from the group consisting of short-acting (regular) insulin, rapid-acting insulin, intermediate-acting (NPH) insulin, and long-acting insulin.

47. The method according to any one of claims 30-46, wherein said treatment comprises maintaining the subject on a low carbohydrate diet.

48. The method according to any one of claims 30-47, wherein said treatment comprise maintaining said subject on a diet that provides a BMI of said subject ranging from about 18.5 to about 24.9.

49. The method according to any one of claims 30-48, wherein said treatment comprise engaging said subject in daily exercise comprising at least 150 minutes of aerobic exercise a week, with no more than two days without any exercise.

50. The method according to any one of claims 30-49, wherein said treatment comprises providing said subject enzyme (ACE) inhibitors or angiotensin II receptor blockers (ARBs).

51. The method according to any one of claims 30-50, wherein said treatment comprises providing said subject aspirin.

52. The method according to any one of claims 30-51, wherein said treatment comprises providing said subject cholesterol lowering drugs.

53. A method of monitoring the onset and/or progression of type 1 diabetes in a subject, said method comprising:
providing a blood sample from said subject at a first time;
performing the method according to any one of claims 1-13 to determine first level(s) of CD57+, CD28−, CD127−, CD27−, CD8+ T cells (SLEC CD8 T cells), and/or CD127−, CD27+, CD57−, CD28− CD8 T cells, and/or CD127−, CD27−, CD57−, CD28− CD8 T cells, and/or CD127+, CD27−, CD57−, CD28− CD8 T cells, and/or associated CD8 T cell subsets shown in FIG. 10, and/or CCR7^{dim}, CD45RA+, CD8+ T cells in said blood sample;
providing a second blood sample from said subject at a second time after said first time;
performing the method according to any one of claims 1-13 to determine second levels of CD57+, CD28−, CD127−, CD27−, CD8+ T cells (SLEC CD8 T cells), and/or CD127−, CD27+, CD57−, CD28− CD8 T cells, and/or CD127−, CD27−, CD57−, CD28− CD8 T cells, and/or CD127+, CD27−, CD57−, CD28− CD8 T cells, and/or associated CD8 T cell subsets shown in FIG. 10, and/or CCR7^{dim}, CD45RA+, CD8+ T cells in said second blood sample, wherein:
an increase in the second levels of CD57+, CD28−, CD127−, CD27−, CD8+ T cells (SLEC CD8 T cells), and/or CD127−, CD27+, CD57−, CD28− CD8 T cells, and/or CD127−, CD27−, CD57−, CD28− CD8 T cells, and/or CD127+, CD27−, CD57−, CD28− CD8 T cells, and/or associated CD8 T cell subsets shown in FIG. 10 compared to the first level(s) of said T cells, and/or a decrease in CCR7^{dim}, CD45RA+, CD8+ T cells compared to the first levels of said T cells, indicates progression of said subject toward type 1 diabetes; and
no increase in the second levels of CD57+, CD28−, CD127−, CD27−, CD8+ T cells (SLEC CD8 T cells), and/or CD127−, CD27+, CD57−, CD28− CD8 T cells, and/or CD127−, CD27−, CD57−, CD28− CD8 T cells, and/or CD127+, CD27−, CD57−, CD28− CD8 T cells, and/or associated CD8 T cell subsets shown in FIG. 10 compared to the first level(s) of said T cells, and/or no decrease in CCR7^{dim}, CD45RA+, CD8+ T cells compared to the first levels of said T cells, indicates little or no progression of said subject toward type 1 diabetes.

54. The method of claim 53, wherein said method comprises determining first and second levels of CD57+, CD28−, CD127−, CD27−, CD8+ T cells (SLEC CD8 T cells).

55. The method according to any one of claims 53-54, wherein said method comprises determining first and second levels of CD127−, CD27+, CD57−, CD28− CD8 T cells.

56. The method according to any one of claims 53-55, wherein said method comprises determining first and second levels of CD127−, CD27−, CD57−, CD28− CD8 T cells.

57. The method according to any one of claims 53-56, wherein said method comprises determining first and second levels of CD127+, CD27−, CD57−, CD28− CD8 T cells.

58. The method according to any one of claims 53-57, wherein said method comprises determining first and second levels of or CCR7^{dim}, CD45RA+, CD8+ T cells in said blood sample.

59. The method according to any one of claims 53-58, wherein said increase in levels and/or said decrease in levels is a statistically significant increase or decrease.

60. The method of claim 59, wherein said increase in level(s) is a statistically significant increase or decrease in level(s) at a p value of p≤0.05, or p≤0.02, or p≤0.01.

61. The method according to any one of claims 53-60, wherein said subject has not progressed to clinical type 1 diabetes.

62. The method of claim 61, wherein said subject shows a normal hemoglobin A1c level ranging from 4% to 5.6%.

63. The method of claim 61, wherein said subject shows an elevated hemoglobin A1c level ranging from 5.7% to 6.4%.

64. The method of claim 61, wherein a random blood sugar test for said subject is below 200 mg/dL (11.1 mmol/L).

65. The method of claim 61, wherein a normal fasting blood sugar level for said subject is less than 100 mg/dL (5.6 mmol/L).

66. The method of claim 61, wherein an elevated fasting blood sugar level for said subject ranges from 100 to 125 mg/dL (5.6 to 6.9 mmol/L).

67. The method according to any one of claims 53-66, wherein said subject is a human and said blood or blood fraction is from said human.

68. A method of treating a subject to slow or prevent the onset of type 1 diabetes, said method comprising:
identifying a subject where the level of CD57+, CD28−, CD127−, CD27−, CD8+ T cells (SLEC CD8 T cells), and/or CD127−, CD27+, CD57−, CD28− CD8 T cells, and/or CD127−, CD27−, CD57−, CD28− CD8 T cells, and/or CD127+, CD27−, CD57−, CD28− CD8 T cells, and/or associated CD8 T cell subsets shown in FIG. 10 in blood or a blood fraction derived from said subject is elevated compared to the level(s) in normal healthy control, and/or or where the level of CCR7^{dim}, CD45RA+, CD8+ T cells in blood or a blood fraction from said subject is reduced compared to the level in a normal healthy control; and
treating said subject to slow or prevent the onset of type 1 diabetes.

69. The method of claim 68, wherein said method comprises identifying a subject where the level of CD57+, CD28−, CD127−, CD27−, CD8+ T cells (SLEC CD8 T cells) is elevated compared to the level in a normal healthy control and treating said subject to slow or prevent the onset of type 1 diabetes.

70. The method according to any one of claims 68-69, wherein said method comprises identifying a subject where the level of CD127−, CD27+, CD57−, CD28− CD8 T cells is elevated compared to the level in a normal healthy control and treating said subject to slow or prevent the onset of type 1 diabetes.

71. The method according to any one of claims 68-70, wherein said method comprises identifying a subject where the level of CD127−, CD27−, CD57−, CD28− CD8 T cells is elevated compared to the level in a normal healthy control and treating said subject to slow or prevent the onset of type 1 diabetes.

72. The method according to any one of claims 68-71, wherein said method comprises identifying a subject where the level of CD127+, CD27−, CD57−, CD28− CD8 T cells is elevated compared to the level in a normal healthy control and treating said subject to slow or prevent the onset of type 1 diabetes.

73. The method according to any one of claims 68-72, wherein said method comprises identifying a subject where the level of CCR7^{dim}, CD45RA+, CD8+ T cells is decreased compared to the level in a normal healthy control and treating said subject to slow or prevent the onset of type 1 diabetes.

74. The method according to any one of claims 68-73, wherein said treatment comprises administering an immune modulator to said subject.

75. The method of claim 74, wherein said immune modulator comprises an anti-viral agent, an anti-PD1 antibody, an anti-CTLA4 antibody, and/or an anti-CD3 antibody.

76. The method of claim 75, wherein said immune modulator comprises an anti-CD3 monoclonal antibody.

77. The method of claim 76, wherein said immune modulator comprises teplizumab.

78. The method of claim 75, wherein said immune modulator comprises an anti-viral agent.

79. The method of claim 78, wherein said immune modulator comprises an anti-viral agent selected from the group consisting of Pleconaril and Ribavirin.

80. The method according to any one of claims 68-79, wherein said method comprises re-induction of tolerance towards the putative self-antigen that causes T1D.

81. The method of claim 80, wherein said antigen comprises one or more antigen selected from the group consisting of insulin, glutamic acid decarboxylase (GAD), and the heat shock protein 60 (Hsp60)-derived peptide 277.

82. The method according to any one of claims 68-81, wherein said treating comprises monitoring blood glucose levels in said subject.

83. The method of claim 82, wherein said blood glucose is tested before meals and snacks, and/or before bed, and/or before exercising or driving.

84. The method of claim 82, wherein said blood glucose is monitored on a daily, a weekly, a biweekly, or a monthly basis.

85. The method according to any one of claims 68-84, wherein said treating comprises fitting said subject with a continuous glucose monitoring system.

86. The method according to any one of claims 68-85, wherein said treating comprises monitoring hemoglobin A1c.

87. The method according to any one of claims 68-86, wherein said treating comprise maintaining normal blood glucose levels.

88. The method of claim 87, wherein said maintaining normal blood glucose levels comprises administering insulin.

89. The method of claim 88, wherein said insulin comprises an insulin selected from the group consisting of short-acting (regular) insulin, rapid-acting insulin, intermediate-acting (NPH) insulin, and long-acting insulin.

90. The method according to any one of claims 68-89, wherein said treating comprises maintaining the subject on a low carbohydrate diet.

91. The method according to any one of claims 68-90, wherein said treating comprise maintaining said subject on a diet that provides a BMI of said subject ranging from about 18.5 to about 24.9.

92. The method according to any one of claims 68-91, wherein said treating comprise engaging said subject in daily exercise comprising at least 150 minutes of aerobic exercise a week, with no more than two days without any exercise.

93. The method according to any one of claims 68-92, wherein said treating comprises providing said subject enzyme (ACE) inhibitors or angiotensin II receptor blockers (ARBs).

94. The method according to any one of claims 68-93, wherein said treating comprises providing said subject aspirin.

95. The method according to any one of claims 68-94, wherein said treating comprises providing said subject cholesterol lowering drugs.

96. The method according to any one of claims 68-95, wherein said subject is a human.

97. A kit for the determination of risk for progression to type 1 diabetes, said kit comprising:
an anti-CD57 antibody, an anti-CD28 antibody, an anti-CD127 antibody, an anti-CD27 antibody, and an anti-CD8 antibody; and/or
an anti-CCR7 antibody, an anti-CD45RA antibody, and an anti-CD8 antibody.

98. The kit of claim 97, wherein said kit comprises an anti-CD57 antibody, an anti-CD28 antibody, an anti-CD127 antibody, an anti-CD27 antibody, and an anti-CD8 antibody.

99. The kit according to any one of claims 97-98, wherein said kit comprises an anti-CCR7 antibody, an anti-CD45RA antibody, and an anti-CD8 antibody.

100. The kit according to any one of claims 97-99, wherein said antibodies are each labeled with a detectable label.

101. The kit of claim 100, where the labels labeling each type of antibody are different and distinguishable.

102. The kit according to any one of claims 100-101, wherein said labels are fluorescent labels.

103. A method of determining if a subject is at risk for developing type 1 diabetes.

104. The method of claim 103, wherein the expression or lack of expression of at least one biomarker is determined, wherein said biomarker may include but is not limited to CD57, CD28, CD127, CD27, CD8, CD4, CD3, Vα7.2. CD45RA, CCR7, CD161, CCR4, FOXP3, CD25, CXCR5, IgG, IgM, and IgA antibodies directed against CMV, CMV RNA, CMV DNA, and CMV proteins.

105. The method of claim 103, wherein specific T cell populations are identified and the presence of said T cell population or lack of said T cell populations are used to determine the relative risk for developing type 1 diabetes.

106. The method of claim 105, wherein the T cell populations may include but are not limited to CD8+, CD57+, CD28−, CD127−, CD27− T cells, CD3+, Vα7.2+, CD161+, CD45RA−, CCR7−, CD28+, CD27−, CD8+/−, CD4+/− T cells, CD3+, Vα7.2+, CD161+, CD45RA^{low}, CCR7^{low}, CD28+, CD128+, CD8+/−, CD4+/− T cells, Vα7.2+. CD161+, CD45RA−, CCR7−, CD28+, CD27−, CD8+/−, CD4+/− T cells, CD4+, CD8−, CD127^{bright}, CXCR5−, CCR4+, CCR7−, CD27+ T cells, CD8+, CD45RA+, CCR7^{dim}, CD57−, CD27+, CD127^{high}, CXCR5−, CCR4− T cells, CD8+, CD127−, CD27−, CD57+, CD28− T cells, CD4+, CD8+/−, CXCR5−, CCR4+, CCR7−, CD27+, CD127^{dim} T cells, CD4+, CD8+/−, CD127^{bright}, CD27+, CCR7−, CCR4+, CXCR5− T cells, CD4+, CCR4+, CXCR5+, CD161− T cells, CD4+, CD8−, CD27−, CCR7+, CCR4+, CXCR5− T cells, CD4+, CD8−, CD27−, CCR7−, CCR4+, CXCR5− T cells, CD4+, CD8−, CCR4+, CXCR5+, CD161− T cells, CD4+, CD8−, CD27−, CCR7+, CCR4+, CXCR5− T cells, CD4+, CD8−, CD27−, CCR7−, CCR4+, CXCR5− T cells, CD4+, CD8−, CCR4+, CXCR5+, CD161− T cells, or combinations thereof.

107. The method of claim 103, wherein the presence of the CMV and other Herpesvirus genome/DNA, CMV RNA, CMV proteins, or IgG, IgM, and IgA antibodies directed against CMV and other Herpesvirus are measured to identify increased risk of development of T1D.

108. The method of claim 107, wherein the CMV and other Herpesvirus markers are measured in conjunction with the T cell markers listed in claim 104 and/or claim 106 to determine a patient's risk of developing T1D.

109. A method of determining if a subject is at risk for developing type 1 diabetes, said method comprising:
a) determining the presence or absence of a specific T cell population in a sample obtained from said subject, wherein the T cell populations may include but are not limited to CD8+, CD57+, CD28−, CD127−, CD27− T cells, CD3+, Vα7.2+, CD161+, CD45RA−, CCR7−, CD28+, CD27−, CD8+/−, CD4+/− T cells, CD3+, Vα7.2+, CD161+, CD45RA^{low}, CCR7^{low}, CD28+, CD128+, CD8+/−, CD4+/− T cells, Vα7.2+, CD161+, CD45RA−, CCR7−, CD28+, CD27−, CD8+/−, CD4+/− T cells, CD4+, CD8−, CD127^{bright}, CXCR5−, CCR4+, CCR7−, CD27+ T cells, CD8+, CD45RA+, CCR7^{dim}, CD57−, CD27+, CD127^{high}, CXCR5−, CCR4− T cells, CD8+, CD127−, CD27−, CD57+, CD28− T cells, CD4+, CD8+/−, CXCR5−, CCR4+, CCR7−, CD27+, CD127^{dim} T cells, CD4+, CD8+/−, CD127^{bright}, CD27+, CCR7−, CCR4+, CXCR5− T cells, CD4+, CCR4+, CXCR5+, CD161− T cells, CD4+, CD8−, CD27−, CCR7+, CCR4+, CXCR5− T cells, CD4+, CD8−, CD27−, CCR7−, CCR4+, CXCR5− T cells, CD4+, CD8−, CCR4+, CXCR5+, CD161− T cells, CD4+, CD8−, CD27−, CCR7+, CCR4+, CXCR5− T cells, CD4+, CD8−, CD27−, CCR7−, CCR4+, CXCR5− T cells, CD4+, CD8−, CCR4+, CXCR5+, CD161− T cells, or combinations thereof, and
b) determining the presence or absence of CMV or other Herpesvirus DNA, CMV RNA, CMV proteins, or IgG, IgM, and IgA antibodies directed against CMV or other Herpesvirus in a sample obtained from said subject.

110. A method of preventing or treating type 1 diabetes in a subject, said method comprising:
a) determining the presence or absence of specific T cell populations in a sample obtained from said subject; and
b) determining the presence or absence of CMV or other Herpesvirus proteins, RNA, DNA, and/or IgG, IgA and IgM antibodies against CMV or other Herpesvirus, and
c) determining an appropriate therapy to prevent and/or treat type 1 diabetes based on the T cell population(s) that were identified; and
d) administering said therapy to said subject.

111. A composition or kit comprising at least one antibody for at least one biomarker selected from the group consisting of CD57, CD28, CD127, CD27, CD8, CD4, CD3, Vα7.2, CD45RA, CCR7, CD161, CCR4, FOXP3, CD25, CXCR5, CMV DNA, CMV or other Herpesvirus RNA, CMV or other Herpesvirus proteins, or IgG, IgA, and IgM antibodies to CMV or other Herpesvirus.

112. The composition or kit of claim 111, wherein said composition or kit also comprises a probe or antibody linked to a probe including but not limited to a fluorescent probe, radiolabeled probe, imaging agent, and/or contrast agent.
